(19) United States
(12) Patent Application Publication
(10) Pub. No.: US 2022/0249158 A1
(43) Pub. Date: Aug. 11, 2022

(54) SYSTEMS AND METHODS FOR LOCALIZED ENDOLUMINAL THERMAL LIQUID TREATMENT

(71) Applicant: Intuitive Surgical Operations, Inc., Sunnyvale, CA (US)

(72) Inventors: ROBERT L. BARRY, Kirkland, WA (US); Erik M. Henne, Seattle, WA (US)

(21) Appl. No.: 17/623,575

(22) PCT Filed: Jul. 7, 2020
(86) PCT No.: PCT/US2020/041075

§ 371 Date: Dec. 28, 2021

Related U.S. Application Data
(60) Provisional application No. 62/871,569, filed on Jul. 8, 2019.
(60) Provisional application No. 62/871,677, filed on Jul. 8, 2019.
(60) Provisional application No. 62/871,678, filed on Jul. 8, 2019.
(60) Provisional application No. 62/938,614, filed on Nov. 21, 2019.
(60) Provisional application No. 62/988,299, filed on Mar. 11, 2020.

(51) Int. Cl. A61B 18/14; A61B 17/12; A61B 18/18; A61B 18/24

(52) U.S. Cl. CPC A61B 18/1492; A61B 17/1204; A61B 17/12104; A61B 17/12109; A61B 2018/0022; A61B 17/12186; A61B 18/1815; A61B 18/24; A61B 17/12136

(57) ABSTRACT

A system for treating target tissue located at a first location along an anatomic lumen comprises a catheter including a distal end portion configured for deployment in the anatomic lumen. The system also comprises an expansion device coupled to the distal end portion of the catheter. The expansion device has an expanded size in a deployed configuration. The expansion device in the deployed configuration occludes the anatomic lumen at a second location, different from the first location, along the anatomic lumen. The expansion device also occludes at least one of a plurality of blood vessels adjacent the anatomic lumen to reduce blood flow to the target tissue at the first location.

### CROSS-REFERENCE TO RELATED APPLICATIONS

This application claims the benefit of U.S. Provisional Application 62/871,569 filed Jul. 8. 2019; U.S. Provisional Application 62/871,677 filed Jul. 8, 2019; U.S. Provisional Application 62/871,678 filed Jul. 8, 2019; 62/938,614 filed Nov. 21, 2019; and U.S. Provisional Application 62/988,299 filed Mar. 11, 2020, all of which are incorporated by reference herein in their entirety.

This application incorporates by reference in its entirety PCT Application (Docket No. P02303-WO) filed Jul. 7, 2020, titled “Systems and Method for Diffuse Endoluminal Thermal Liquid Treatment.”

### FIELD

Examples described herein are related to systems and methods for localized endoluminal thermal treatment of diseased anatomy.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments such as therapeutic instruments, diagnostic instruments, imaging instruments, and surgical instruments. In some examples, a minimally invasive medical instrument may be a thermal energy treatment instrument for use within an endoluminal passageway of a patient anatomy.

### SUMMARY

The following presents a simplified summary of various examples described herein and is not intended to identify key or critical elements or to delineate the scope of the claims.

In some examples, a system for treating target tissue located at a first location along an anatomic lumen may comprise a catheter including a distal end portion configured for deployment in the anatomic lumen. The system may also comprise an expansion device coupled to the distal end portion of the catheter. The expansion device has an expanded size in a deployed configuration. The expansion device in the deployed configuration may occlude the anatomic lumen at a second location, different from the first location, along the anatomic lumen. The expansion device may also occlude at least one of a plurality of blood vessels adjacent the anatomic lumen to reduce blood flow to the target tissue at the first location.

In some examples, a system may comprise a processor and a memory having computer readable instructions stored thereon. The computer readable instructions, when executed by the processor, may cause the system to expand a treatment device positioned within a first anatomic lumen at a first location along a length of the first anatomic lumen and apply heat using the treatment device to occlude a first blood vessel adjacent to the first anatomic lumen to reduce blood flow to a target tissue. The target tissue may be at a second location along the length of the first anatomic lumen and the second location may be at a different location than the first location.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1A is a simplified diagram of a patient anatomy according to some examples.

FIG. 1B illustrates a region of the patient anatomy of FIG. 1A according to some examples.

FIG. 1C is a cross-sectional view of a region of the patient anatomy of FIG. 1A according to some examples.

FIG. 2 is a flowchart illustrating a method for applying a thermal energy treatment to an endoluminal passageway to occlude an adjacent blood vessel, according to some examples.

FIG. 3 is a detailed view of a portion of the patient anatomy of FIG. 1A with a treatment instrument within an anatomic lumen, according to some examples.

FIG. 4 is a detailed view of a portion of the patient anatomy of FIG. 3 with an expanded treatment instrument in the anatomic lumen, according to some examples.

FIG. 5 is a flowchart illustrating a method for applying a thermal energy treatment to a surface of an endoluminal passageway, according to some examples.

FIGS. 6A and 6B are detailed views of a portion of the patient anatomy of FIG. 1A with a treatment instrument within the anatomic lumen, according to some examples.

FIGS. 7A-1 and 7A-2 are cross-sectional views of a region of the patient anatomy treated for chronic bronchitis, according to some examples.

FIGS. 7B-1 and 7B-2 are cross-sectional views of a region of the patient anatomy treated for bronchiectasis, according to some examples.

FIGS. 7C-1 and 7C-2 are cross-sectional views of a region of the patient anatomy treated for emphysema, according to some examples.

FIGS. 7D-1 and 7D-2 are cross-sectional views of a region of the patient anatomy treated for emphysema, according to some examples.

FIGS. 7E-1 and 7E-2 are cross-sectional views of a region of the patient anatomy treated for a lung tumor, according to some examples.

FIG. 8 illustrates a medical instrument with an expandable device, according to some examples.

FIG. 9 illustrates a medical instrument with an expandable device, according to some examples.

FIG. 10 illustrates a medical instrument with an expandable device, according to some examples.

FIG. 11 illustrates a medical instrument with an expandable device, according to some examples.

FIG. 12 illustrates a medical instrument with an expandable device, according to some examples.

FIG. 13 illustrates a medical instrument with an expandable device, according to some examples.

FIG. 14 illustrates a medical instrument with an expandable device, according to some examples.

FIG. 15 illustrates an expandable device, according to some examples.

FIG. 16 illustrates a medical instrument with an expandable device, according to some examples.

FIG. 17 is a flowchart illustrating a method for occluding an artery with access via an adjacent bronchial passageway, according to some examples.

FIG. 18 illustrates an arterial occlusion device according to some examples.

FIG. 19 illustrates an arterial occlusion device according to some examples.

FIG. 20 illustrates an arterial occlusion device according to some examples.

FIG. 21 illustrates an arterial occlusion device according to some examples.

FIGS. 22A and 22B illustrate an arterial occlusion device according to some examples.

FIG. 23 illustrates a robot-assisted medical system according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The technology described herein provides techniques and treatment systems for endoluminal thermal treatment of diseased tissue. Although the examples provided herein may refer to treatment of lung tissue and pulmonary disease, it is understood that the described technology may be used in treating artificially created lumens or any endoluminal passageway or cavity, including in a patient trachea, colon, intestines, stomach, liver, kidneys and kidney calices, brain, heart, circulatory system including vasculature, fistulas, and/or the like. In some examples, treatment described herein may be referred to as endobronchial thermal liquid treatment and may be used in procedures to treat lung tumors and/or chronic obstructive pulmonary disease (COPD).

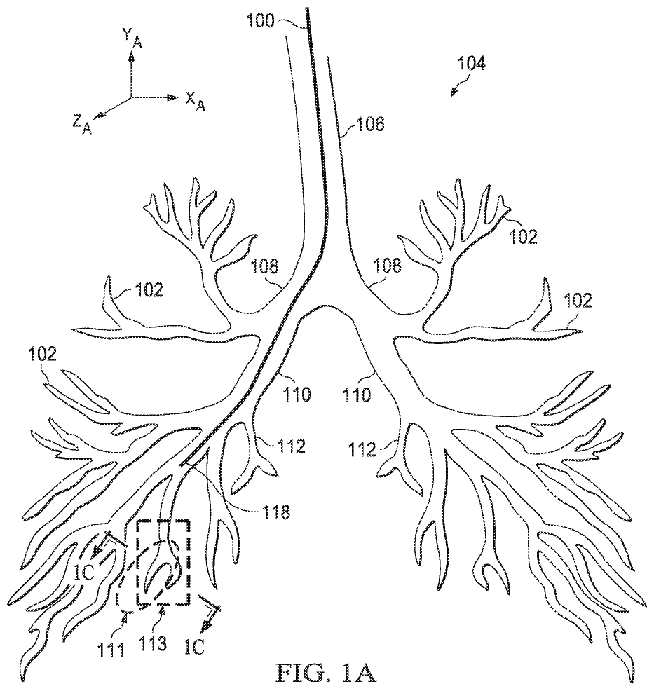

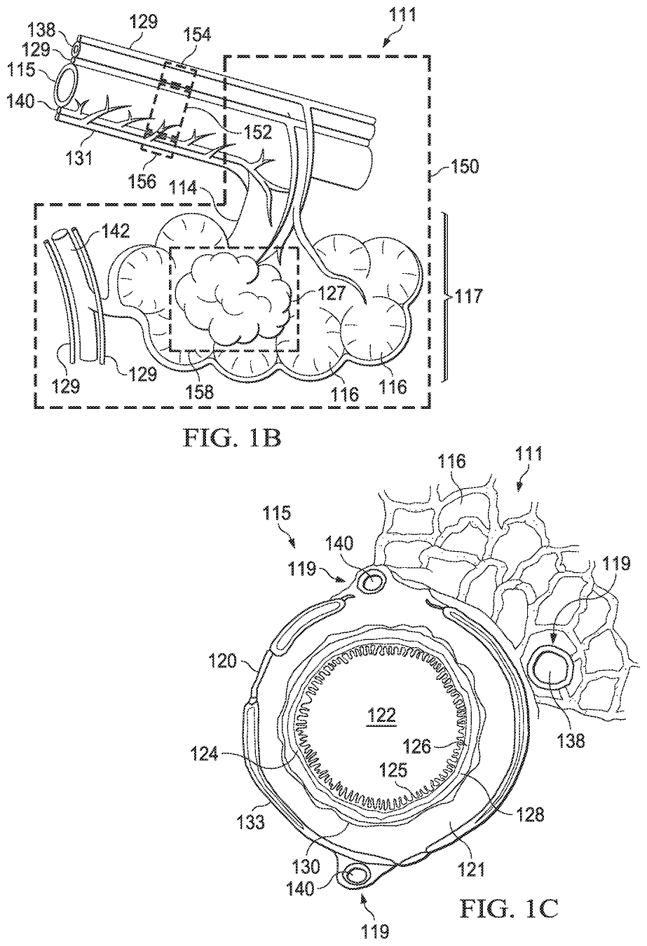

FIG. 1A, illustrates an elongated medical instrument system 100 extending within branched anatomic passageways or airways 102 of an anatomical structure 104. In some examples the anatomic structure 104 may be a lung and the passageways 102 that include the trachea 106, primary bronchi 108, secondary bronchi 110, and tertiary bronchi 112. The anatomic structure 104 has an anatomical frame of reference (X_{A}, Y_{A}, Z_{A}). A distal end 118 of the medical instrument 100 may be advanced into an anatomic opening (e.g. the mouth) and through the anatomic passageways 102 to perform a medical procedure, such as a thermal energy treatment, at or near target tissue located in a region 113 of the anatomic structure 104. As shown in FIG. 1B, a distal-most region 111 of the branched anatomic passageways, which may be distal of the tertiary bronchi 112, may include a small bronchus 115 and the lung parenchyma 117, including bronchioles 114 and alveoli 116, which is involved in gas exchange. Vasculature 119 extending along the bronchus 115 may include a pulmonary artery 138 and a bronchial artery 140. A pulmonary vein 142 transfers oxygenated blood from the lungs to the heart. Nerves 131 and lymphatic vessels 129 may also extend within the region 111. Target tissue 127, such as a tumor, may be formed in the parenchyma 117.

FIG. 1C illustrates across-sectional view of the bronchus 115 that includes a lumen 122 defined by an inner bronchial wall 124. The inner diameter of the bronchial wall 124 may be lined by an epithelium layer 126 which includes goblet cells. Mucocilia 125 are tiny hairs that extend from the epithelium 126 into the lumen 122. The epithelium 126 may be surrounded by a lamina propria layer 128 which may be surrounded by a smooth muscle layer 130. A sub mucosa layer 121 surrounds the smooth muscle layer 130, and a layer of continuous or discontinuous cartilage 133 covers the sub mucosa layer. A connective tissue layer of adventitia 120 may surround and provide support to the bronchus 115. The vasculature including the bronchial artery 140, the pulmonary artery 138, and the pulmonary vein 142 may extend along the bronchus 115 to supply blood flow to and from the lung region.

Lung tumors, such as tumor 127, may include ground glass opacity tumors, semi-solid tumors, or spiculated tumors. Often, they may occur in the peripheral, outer third of the lung volume. Some lung tumor treatment methods and systems described herein may be particularly suited for the low-density, peripheral areas of the lung. Tumors may be cancerous, and effective treatment of cancer in the lung may include the destruction of the tumor core, peripheral tumors, and cancerous or non-cancerous areas at the margin of the tumors. Conventional ablation treatment may directly heat the tumor core without treating the tissue at the margin. For some cancers, a more effective treatment may infarct a segment of the lung, thus destroying tumors and the tissue around the tumor within the infarcted region. As shown in FIG. 1B, the arteries 140, 138 may sustain and support the growth of tumor 127. Conventional ablation treatments may apply heat directly to the tumor 127, leaving the parenchyma 117 and the surrounding vasculature and bronchial structures generally intact. As described in detail below, for some tumors or other lung conditions, infarction of a segment 150 including the tumor 127 by applying localized heating to the bronchus 115 and/or the surrounding vasculature (138, 140) may destroy not only the tumor 127 but also the tissue at the margins of the tumor and the vasculature that delivers oxygen and supports the growth and potential regrowth of the tumor. In some embodiments, reliable lung tumor elimination may be achieved by simultaneous occlusion of multiple supporting structures, including the bronchus 115, the pulmonary artery 138 and/or the bronchial artery 140. For example, as described below, a heated balloon deployed within the bronchial lumen at a location 152 may compress or reduce the diameter of the pulmonary artery 138 at a location 154 to restrict heat-dissipating blood flow to a location 158 that includes the tumor 127. Additionally, the heated balloon may heat the bronchus 115 at the location 154 to generate scarring and occlusion at the location 152. Further, the heated balloon within the bronchus 115 may heat the bronchial artery 140 at a location 156 to occlude the artery 140. The heated balloon may also compress the bronchial wall, placing the heat source closer to the arteries to more efficiently deliver ablative heat to the arterial locations 154, 156. Conventional techniques that may deliver endoluminal treatment through the arteries to directly ablate only the arteries may not simultaneously occlude multiple structures such as the pulmonary artery, the bronchial artery, and the bronchus. Occluding multiple structures with a single treatment may more efficiently and effectively infarct an entire lung segment 150 surrounding a tumor 127.

The systems and techniques described herein may also be deployed in the treatment of chronic obstructive pulmonary disease that may include one or more of a plurality of disease conditions including chronic bronchitis, emphysema, and bronchiectasis. Chronic bronchitis is the inflammation of bronchial tubes and may be characterized by increased mucous secretions, infections and exacerbations from goblet cell hyperplasia. Emphysema is a condition in which the alveoli at the distal ends of the bronchial tubes are damaged resulting in poor gas exchange and hyperinflation of alveoli, thereby causing reduced pulmonary function. Bronchiectasis is a condition in which the bronchial tubes become widened and thickened by scarring. Pockets form in the bronchial tube walls, creating a nidus for bacterial biofilm, infections and bronchiectasis exacerbations.

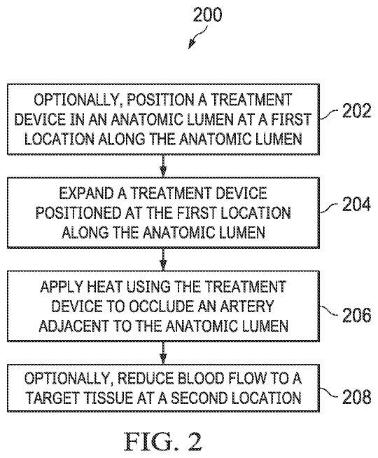

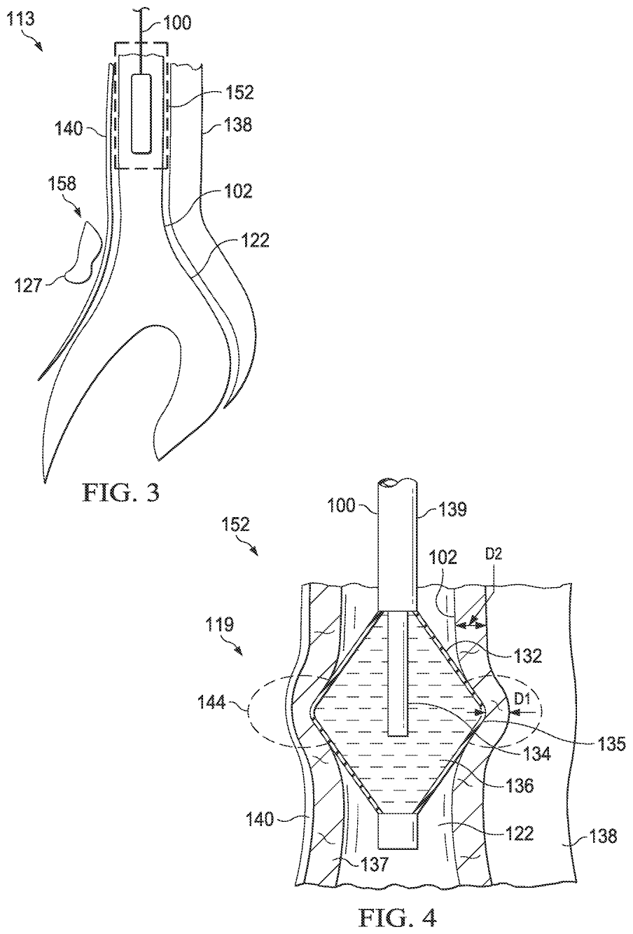

FIG. 2 is a flowchart illustrating a method 200 for applying a thermal energy treatment to an endoluminal passageway to occlude one or more adjacent blood vessels. The method 200 is illustrated as a set of operations or processes that may be performed in the same or in a different order than the order shown in FIG. 2. One or more of the illustrated processes may be omitted in some embodiments of the method. Additionally, one or more processes that are not expressly illustrated in FIG. 2 may be included before, after, in between, or as part of the illustrated processes. In some embodiments, one or more of the processes of method 200 may be implemented, at least in part, by a control system executing code stored on non-transitory, tangible, machine-readable media that when run by one or more processors (e.g., the processors of a control system) may cause the one or more processors to perform one or more of the processes. Reference to FIGS. 3-4 will be made to further illustrate the processes of method 200.

At an optional process 202, a treatment device is positioned in an anatomic lumen at a first location. For example, and with reference to FIG. 3 which provides a detailed view of the region 113 of the lung 104, a treatment device in the form of medical instrument 100 may be positioned at a first location 152 in a lumen 122 of anatomic passageway 102. Pulmonary blood vessels or vasculature 119 may extend alongside the bronchial passageway 102. As shown in greater detail in FIG. 4, the pulmonary vasculature 119 extends along a bronchus wall 137 and may include a pulmonary artery 138 and a bronchial artery 140. A target tissue 127, which may be, for example a lung tumor, may be located at a second location 158 distal of or downstream from the first location 152. In some embodiments, the lung tumor may have a diameter of approximately 2 cm or smaller, although the methods described herein may also be used for larger tumors. The positioning of the medical instrument 100 may be performed with a robot-assisted endoluminal medical system or may be performed with an endoscope manually by a clinician. In some embodiments, a determination of the first location may be made based on a pre-determined treatment distance from the second location. In some embodiments, a robot-assisted endoluminal medical system may use imaging data or a user input to identify the second location and may then use the imaging data or a user input to determine the first location. For example, the first location may be identified based on a predetermined distance from second location, an analysis of the blood supply to the second location, pre-operative or intra-operative imaging, and/or based on proximity of the vasculature and airways. In some embodiments, pre-operative or intra-operative imaging may be used to measure or determine an anatomical dimension such as a distance from airway to artery, a lumen diameter, and/or a radial or circumferential location of the artery in in relation to the airway orientation (e.g. at 10 o'clock). The anatomical dimensions may be used to select a size of the expandable device or plan the extent of expansion of the expandable device. In some embodiments, blood flow sensor data from, for example, pressure sensors, force sensors, impedance sensors, and/or live imaging or other vision-based systems may be used to detect blood flow.

At a process 204, the treatment device is expanded at the first location to dilate the anatomic lumen and compress adjacent vasculature. For example, and with reference to FIG. 4, the medical instrument 100 may be include a flexible catheter 139 with an expansion or expandable device 132 coupled to a distal end of the catheter. The expandable device 132 may be, for example, a balloon filled with a liquid 136 or other non-compressible fluid. Thus, in this embodiment, the medical instrument 100 may be referred to as a heated balloon catheter system. The expandable device 132 may be expanded by the introduction of the liquid into the expandable device or may be expanded by an inflation fluid and subsequently filled with the liquid 136 after expansion. The liquid may be provided from a liquid source or reservoir coupled to a proximal end of the catheter. The liquid 136 within the expandable device 132 may be a non-compressible fluid such as water, saline, gel, or oil. In some embodiments, the expandable device may be generally compliant and may conform to the airway walls of the anatomic passageway 102. In other embodiments, the expandable device may be generally non-compliant and may maintain a predetermined expanded configuration that creates a dilation effect on passageways with a diameter smaller than the diameter of the expandable device. In other embodiments, the expandable device may be generally semi-compliant and may create a partial dilation effect on passageways with a diameter smaller than the diameter of the expandable device. In some embodiments, the expandable device may include compliant, semi-compliant, and/or non-compliant regions. In other embodiments, the expandable device may be an expandable scaffolding, a stent, or other expanding device. In some embodiments, the expandable device may expand to a predetermined size and configuration. In some embodiments, the first location may be determined real-time, during the procedure. For example, the instrument 100 may be positioned near the target tissue (e.g. the tumor 127), and then, while monitoring blood flow, the instrument may be retracted, following the blood flow, to a proximal location (e.g., the first location) from the tumor the expandable device. The monitoring may be performed with a control system (e.g., a control system 512) in receipt of blood flow sensor data from, for example, pressure sensors, force sensors, impedance sensors, and/or live imaging or other vision-based systems to detect blood flow.

In the expanded configuration, the expandable device 132 may have a generally diamond-shaped cross-section including a ridge 135 that causes bronchial dilation and a localized compression of the vasculature 119 near or adjacent to the first location 152. The bronchus wall 137 and surrounding connective tissue, lymphatic tissue, and neurological tissue may also be compressed by the expandable device. For example, the pulmonary artery 138 may be narrowed or compressed, thus restricting or blocking blood flow through the compressed region and preventing or limiting distal blood flow to the target tissue 127 at the second location 158. This acute blockage of blood flow may reduce or eliminate the heat dissipation that would otherwise occur with flowing blood. Because heat is not dissipated by the blood flow, the size of a heated region 144 may increase and ultimately occlusive neointima hyperplasia may result. Because the expandable device 132 expands the lumen 122, a compressed distance D1 of the bronchus wall 137 between the inner wall of lumen 122 and the pulmonary artery 138 may be smaller compared to a distance D2 at an uncompressed location. In some embodiments, the extent of the expansion of the expandable device may be controlled based, for example, on the diameter of the passageway and the desired amount of compression on surrounding tissue. In some embodiments, the extent of the expansion may be monitored and/or controlled by a robot-assisted medical system control system (e.g. control system 512). For example, the control system may control expansion based on sensor feedback including sensed pressure, sensed force, measured impedance for tissue contact, and live imaging or other vision-based methods (e.g., fluoroscopic imaging or CT imaging) to detect arterial compression. Imaging and vision-based methods may also be used to monitor expansion of a diameter of the expandable device. In some embodiments, when a non-compliant balloon is used as the expandable device, a measured volume of liquid may be used to monitor and determine the expanded diameter of the expandable device.

In some embodiments, the uncompressed distance D2 between the bronchus wall and the vasculature may be determined based on sensor data including internal imaging source data (e.g., ultrasound sensor data, other imaging sensor data) or external imaging source data (e.g., pre-operative or intra-operative CT data or MRI data), or may be determined based on the airway generation of the first location and pre-determined standardized model estimates of the distance at the particular airway generation. In some embodiments, if the diameter of the bronchus and the uncompressed distance D2 are determined, the expansion may be based on the ratio of the diameter to the distance D2. In some embodiments, the distance may be evaluated and determined by a control system of a robot-assisted medical system (e.g., control system 512). In some embodiments, the expanded configuration of the treatment device may be determined based on the determined uncompressed distance. For example, if the determined distance is relatively small (e.g., the artery is close to the airway), the expandable device may be expanded to a relatively narrow diameter, providing only light compression. By contrast, if the determined distance is relatively large (e.g., the artery is farther from the airway), the expandable device may be expanded to a larger diameter, providing more compression. In some embodiments, the control system and one more monitoring sensors may comprise a closed-loop system that may prevent over-expansion of the expandable device. For example, the expansion may be limited based on a monitored expansion of the airway diameter or based on a measured pressure. In some embodiments, if the measured distance D2 from the passageway to the artery exceeds a threshold distance or if a monitored dilation diameter exceeds a threshold diameter, the expansion procedure may be suspended, or an indication may be provided to a user or control system to stop expansion to prevent rupturing the passageway.

At a process 206, heat is applied using the treatment device to occlude an artery adjacent to the anatomic lumen. For example, a heating system 134 within the expandable device 132 may heat the liquid 136. The heat may be conducted through the bronchus wall 137 to the vasculature 119 to cause an occlusion of the vasculature. Within a heated region 144, the pulmonary artery 138, for example, may develop an arterial occlusion caused by a thrombosis, scar tissue, or collagen contraction, or other reaction of the anatomy to the applied heat. In some examples, the occlusion may occur entirely during the application of the heat via thrombosis, and in other examples, the occlusion may develop over a period of time as the anatomy responds to the injury caused by the heat. For example, neointima hyperplasia is a type of scarring that may occur four to six weeks following the process 206. In some examples, the heat may also cause scarring of the airway lumen 122 which may occlude or restrict airflow through the lumen 122. In some examples, the bronchial artery 140 within the heated region 144 may also be occluded. In some examples, the heat may also destroy connective tissue, lymphatic tissue, parenchymal tissue, and/or nerves adjacent to or near the location 152. In some embodiments the temperature of the liquid 136 may be monitored and regulated using a control system of a robot-assisted medical system (e.g., a control system 512).

At an optional process 208, blood flow to the target tissue at the second location may be reduced. For example, the occlusion of the pulmonary artery 138 may cause apoptosis of distal (e.g., downstream) cells, anoxia, and infarction of the tissue distal of the first location supplied by the artery 138, including the second location 158. Because the tissue may be destroyed via anoxia or non-thermal fixation, over time coagulative necrosis of the infarcted tissue, including the target tissue 127 and the surrounding margin may occur, followed by macrophage removal of the necrotic tissue. By contrast, thermal fixation caused by direct ablation may cause necrosis but the necrotic tissue may not be removed by the macrophage process. As compared direct ablations (e.g., using radio frequency ablation, microwave ablation, or stereotactic body radiation therapy) that cause thermal fixation of tissue, anoxia and infarction allows the immune system to recognize the antigens of the dead cancer cells and allows for an immuno-stimulation effect that can stimulate the immune system to target other tumors in the body through the abscopal effect.

In some embodiments, blood flow through the artery 138 may be monitored at the first location or at a downstream location to measure the effectiveness of the occlusion in reducing blood flow. The monitoring may be performed with a control system (e.g., a control system 512) in receipt of blood flow sensor data from, for example, pressure sensors, force sensors, impedance sensors, and/or live imaging or other vision-based systems to detect blood flow.

In some embodiments, post-operative imaging (e.g., CT or MRI imaging) may be used to determine if a thrombosis has occurred. In some embodiments, intra-operative imaging, such as endobronchial ultrasound may be used to measure anatomical dimensions such as distance from airway to artery or lumen diameter.

In some embodiments, a second artery providing blood to the target tissue and a second anatomic lumen adjacent the second artery may be determined. The processes 202-208 may be repeated within the second anatomic lumen to further reduce blood flow to the target tissue and promote ischemia of the target tissue. Additional supply arteries and adjacent anatomic lumens may be identified and treated with the heated expandable device until the blood flow to the target tissue is fully obstructed.

In some embodiments, a total acute collapse of the pulmonary artery, via compression and heat from the expansion device, may not be required to achieve total permanent occlusion of the pulmonary artery. This is because heat applied to the inner diameter of the pulmonary artery closest to the heated dilated balloon may cause platelet activation and aggregation which may be sufficient to create acute occlusion.

In some embodiments, reliable lung tumor removal may be achieved by occlusion of the bronchial lumen, the pulmonary artery and the bronchial artery as described in method 200 and the various alternative embodiments. In some embodiments, a diffuse heated liquid treatment described in PCT Application (Docket No. P02303-WO) filed Jul. 7, 2020, titled “Systems and Method for Diffuse Endoluminal Thermal Liquid Treatment” and incorporated by reference herein in its entirety may be further used, in addition to method 200, to perform ablation of the pulmonary artery, the bronchial artery, and/or the bronchial passageway. In some embodiments, the expandable device 132 may not occlude all of the pulmonary artery, the bronchial artery, and the bronchial passageway. For example, the heat from the expandable device may cause occlusion of the bronchial artery and the bronchial passageway but may cause no or incomplete occlusion of the pulmonary artery. As described in embodiments below, targeted ablation of the pulmonary artery may be performed by penetrating the bronchial wall with an arterial occlusion device to directly occlude the pulmonary artery. In some embodiments, the target tissue 127 may be directly ablated either before or after occlusion of the bronchial lumen, the pulmonary artery and/or the bronchial artery as described in method 200 and the various alternative embodiments. Any one or combination of the bronchial passageways 102, the vasculature 119, the parenchyma 117, and/or the target tissue 127 may be treated simultaneously or serially using any of the methods, systems, or devices described herein.

In some embodiments, the method 200 may be suitable for lung tumors having a diameter of approximately 2 mm or smaller. In other embodiments, the method 200 may be suitable for larger tumors. Optionally, either before, after or during the processes 204-208, the target tissue 127 may be directly ablated, for example by a radiofrequency (RF) or microwave (MW) ablation probe, ultrasound, cryotherapy, chemical ablation, or direct heat. For example, an RF or MW ablation probe may directly ablate the core of a lung tumor 127 before, during or after inducing the segmental infarction of the second location 158. Optionally, the expandable device may be moved from the first location to the second location to directly ablate the target tissue. Optionally, the treatment device may be a telescoping device that compresses and occludes the artery at the first location as described and subsequently or simultaneously has an extended catheter member that extends to the second location to directly ablate the target tissue.

Although the method 200 has been described with reference to the target tissue being a lung tumor, in other embodiments, the target tissue may be emphysematous lung tissue or other tissue afflicted with other diseases. Emphysematous segments of the lung may conduct gas exchange poorly and may be hyperinflated such that the diseased segments encroach on healthier lung segments, diminishing pulmonary function and quality of life. The methods described above for airway and arterial occlusion may be used to cause infarction of emphysematous lung segments and reduce emphysema in the lungs resulting in improved pulmonary function and quality of life.

In some embodiments, the liquid 136 may be heated to a temperature of less than 100 degrees Celsius at a sea level pressure to maintain a liquid form, without transitioning to a gaseous phase. In some embodiments, higher temperatures may be useful to ensure that the vasculature, including the regions farthest from the expandable device, are sufficiently heated to result in occlusion. Thus, in some embodiments, the temperature of the liquid in the expandable device, at pressures higher than sea level, may be greater than 100 degrees Celsius. For example, if the liquid is water and a pressure of 20 atm is reached in the expandable device, the temperature of the liquid may be heated to a temperature of less than 215 degrees Celsius before the water transitions to a vapor. In some embodiments, oils or other liquids may be used to achieve higher temperatures at the expandable device. When the temperature of the liquid is greater than 100 degrees Celsius, secondary effects, such as thermal fixation, may occur in the anatomic passage. This may be acceptable, for example in the case of lung tumor treatment, when the objective is the infarction of a lung segment that includes the lung tumor.

In some embodiments, the process 204 may be altered or omitted such that the treatment device does not compress the adjacent vasculature. For example, if the vasculature is sufficiently close to the airway or if the wall of the airway is sufficiently thin, the heated liquid within the expandable device may be conducted to the adjacent vasculature and may occlude the adjacent vasculature without compression. The bronchial artery, for example, may be sufficiently close to the bronchus and may have a sufficiently small diameter that the expandable device may provide enough heat to cause platelet aggregation and subsequent occlusion.

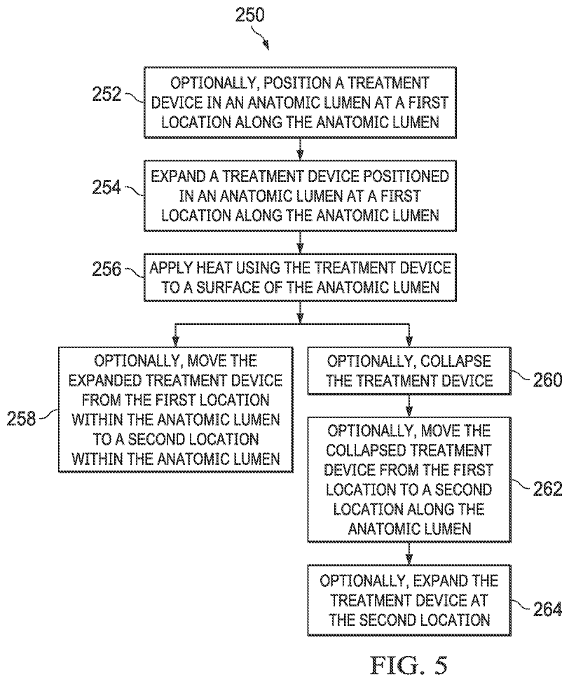

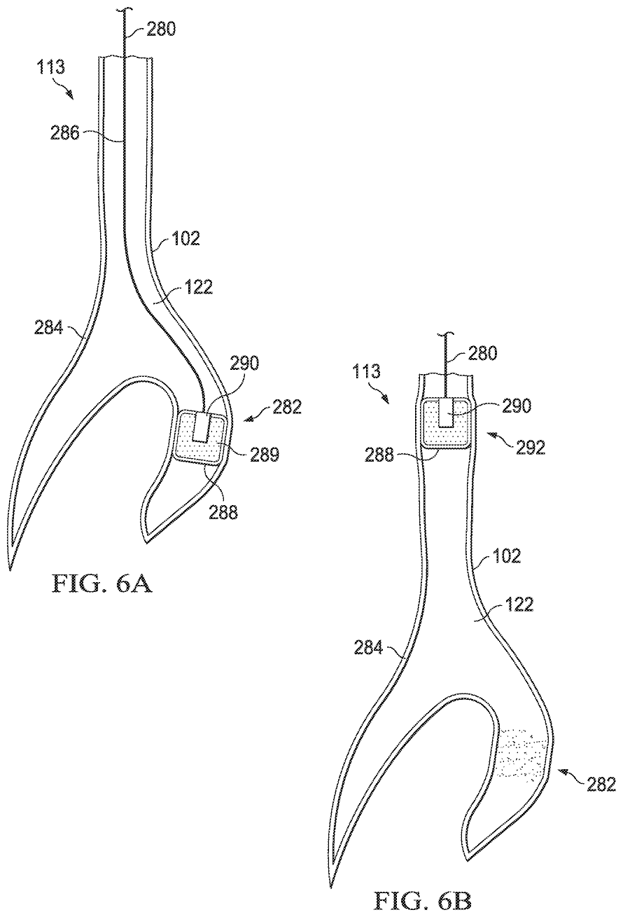

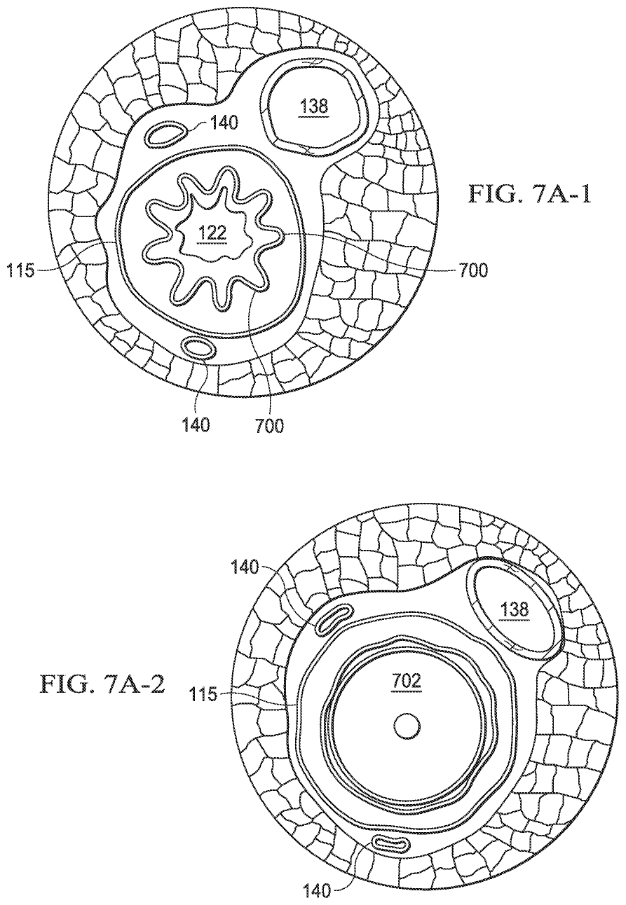

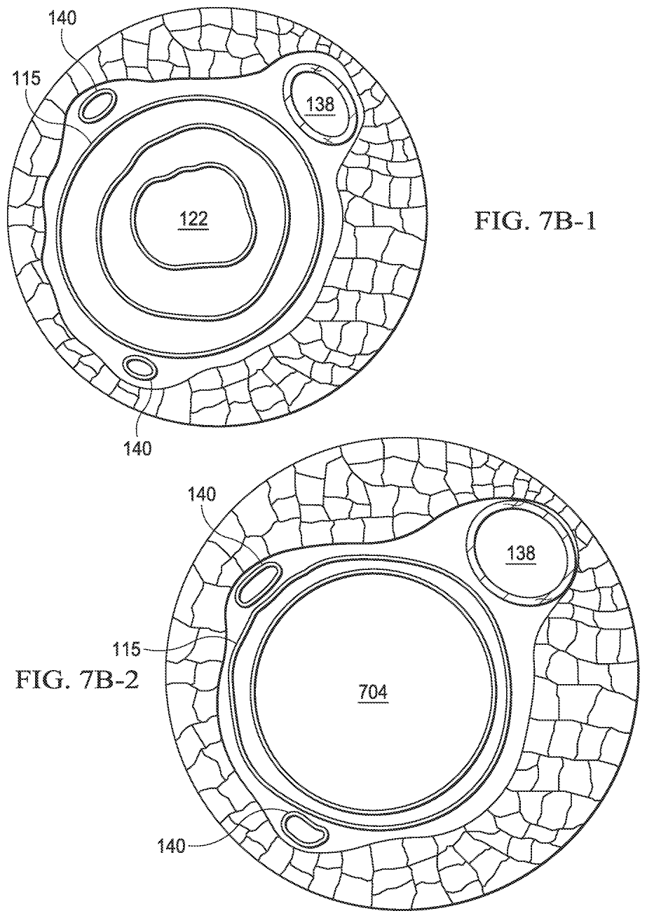

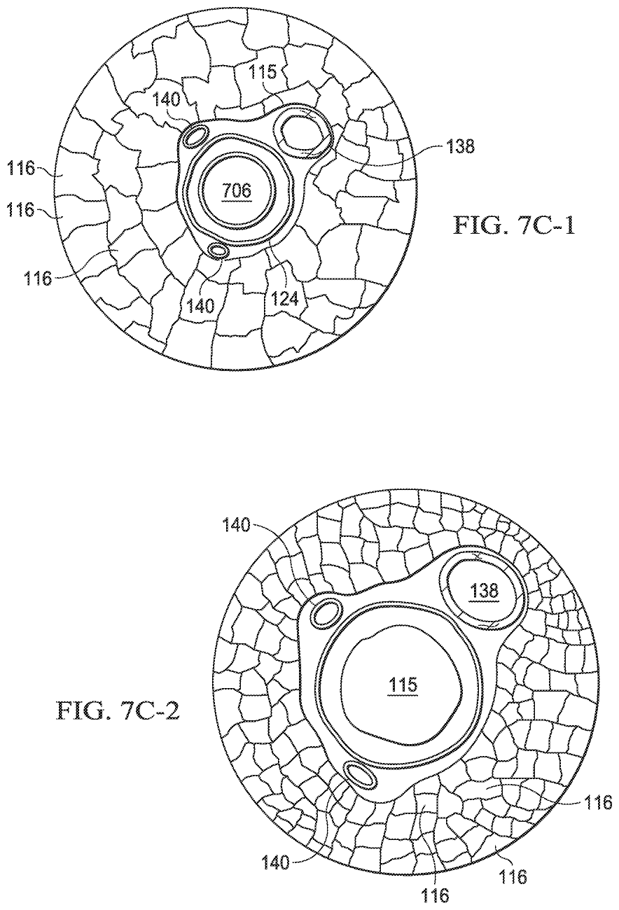

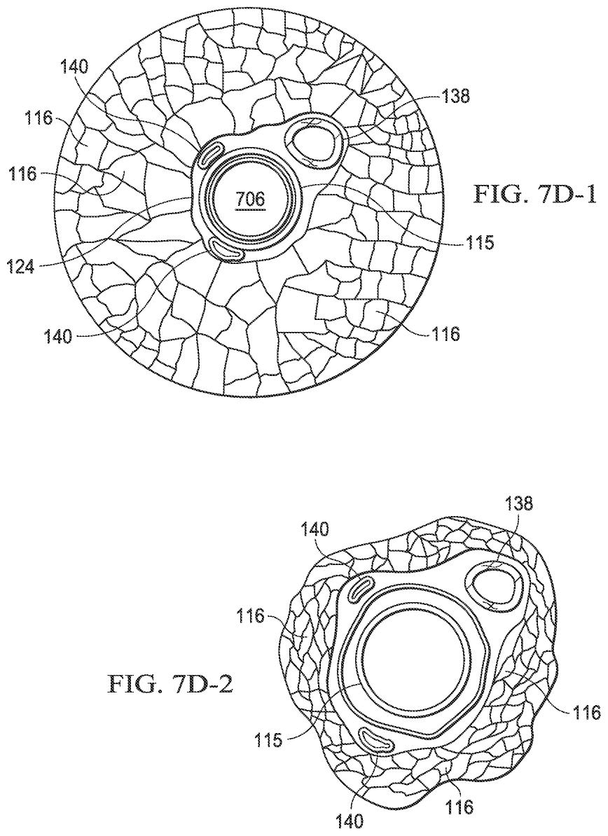

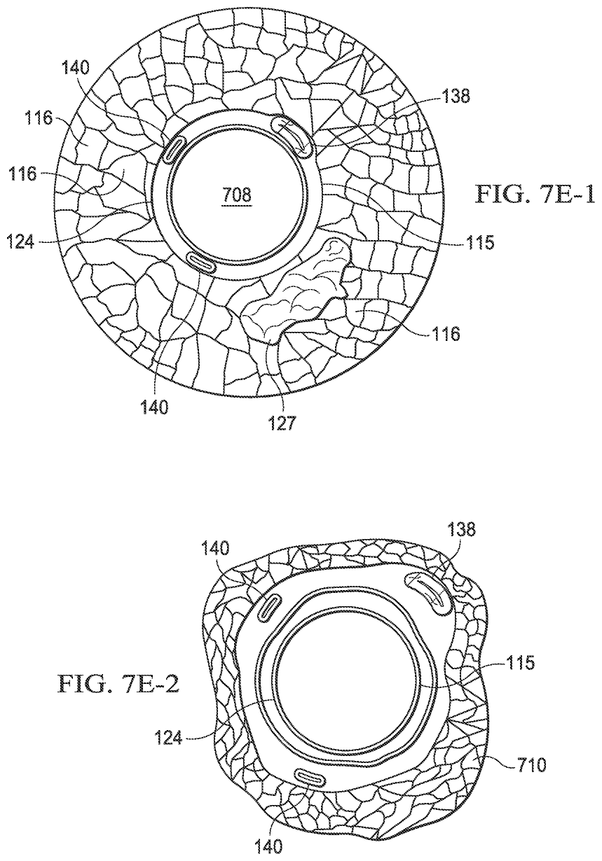

FIG. 5 is a flowchart illustrating a method 250 for applying a thermal energy treatment to ablate an endoluminal passageway. The method 250 is illustrated as a set of operations or processes that may be performed in the same or in a different order than the order shown in FIG. 5. One or more of the illustrated processes may be omitted in some embodiments of the method. Additionally, one or more processes that are not expressly illustrated in FIG. 5 may be included before, after, in between, or as part of the illustrated processes. In some embodiments, one or more of the processes of method 250 may be implemented, at least in part, by a control system executing code stored on non-transitory, tangible, machine-readable media that when run by one or more processors (e.g., the processors of a control system) may cause the one or more processors to perform one or more of the processes. Reference to FIGS. 6-7 will be made to further illustrate the processes of method 250.

At an optional process 252, a treatment device is positioned in an anatomic lumen at a first location. For example, and with reference to FIG. 6A which provides a detailed view of the region 113 of the lung 104, a treatment device in the form of a medical instrument 280 may be positioned at a first location 282 in the lumen 122 of anatomic passageway 102. In this example, target tissue 284 may be located throughout the region 113. The target tissue may be, for example lung tissue with chronic bronchitis, emphysematous tissue, tissue diseased with bronchiectasis, parenchymal tissue, or hemorrhagic tissue. The positioning of the medical instrument 280 may be performed with a robot-assisted medical system or may be performed manually by a clinician. In some embodiments, the first location 282 may be distal of a planned second location 292.

At a process 254, the treatment device is expanded at the first location. For example, and with reference to FIG. 6A, the medical instrument 280 may include a flexible catheter 286 with an expansion or expandable device 288 coupled to a distal end of the catheter. The expandable device 288 may be, for example, a balloon filled with a liquid 289 or other non-compressible fluid. The liquid may be a non-compressible fluid such as water, saline, gel, or oil. In some embodiments, the liquid 289 may be used to inflate the balloon, but in other embodiments, the balloon may be expanded by mechanical means or by the introduction of another fluid. In the expanded configuration, the expandable device 288 may physically contact the airway walls of lumen 122. In some embodiments, the expandable device may be generally compliant and may conform to and radially contact the airway walls of the anatomic passageway 102. In other embodiments, the expandable device may be generally non-compliant and may maintain a predetermined expanded configuration. In some embodiments, the expandable device may include compliant and non-compliant regions. In some embodiments, the expandable device may touch the anatomic passageway wall with no or insignificant dilation of the passageway. Alternatively, the expandable device may apply pressure to the anatomic passageway wall to dilate the lumen.

At a process 256, heat is applied to the surface (and optionally the sub-surface) of the wall of the anatomic lumen using the heated treatment device. For example, a heating system 290 within the expandable device 288 may heat the liquid 289 and cause ablation of the target tissue 284 in the anatomic passageway 102. The depth of ablation and therefore the anatomical structures (e.g., bronchial passageway, bronchial artery, pulmonary artery, etc.) occluded by the ablation may be controlled based on the duration of application, the temperature of the expanded and heated device, and the determined distance between the heated, expandable device and the anatomical structure. In some embodiments, the distance between the bronchial wall and the vasculature at the first location may be determined based on sensor data including internal imaging source data (e.g., ultrasound sensor data, other imaging sensor data) or external imaging source data (e.g., pre-operative or intra-operative CT data or MRI data), or may be determined based on the airway generation of the first location and pre-determined standardized model estimates of the distance at the particular airway generation.

Ablation may induce cellular and structural changes in the epithelium that in some cases may extend to the sub-epithelium. The ablation may cause tissue reduction, including destruction of goblet cells and cilia at the first location 282. In some embodiments, the cellular matrix may be preserved to allow for later regrowth of healthy cells. The size of the ablation region, depth of ablation, and tissue structures affected by the ablation may be varied based on time of contact with the expandable device 288, amount of material interface between the expandable device 288 and the tissue, contact pressure, and the temperature of the liquid and expandable device. In some examples, the tissue reaction may occur entirely during the application of the heat, and in other examples, the tissue damage may develop over a period of time as the anatomy responds to the injury caused by the heat. As described above, in some examples, the liquid 289 may be heated to a temperature of less than 100 C° at sea level, but in other examples the temperature of the liquid may be greater than 100 C°.

At an optional process 258, the treatment device, while in the expanded configuration, may be moved from the first location within the anatomic lumen to a second location within the anatomic lumen. For example, and with reference to FIG. 6B, the medical instrument 280 may be moved from the first location 282 to a second location 292, dragging the expandable device 288 along the anatomic passageway 102. With the expandable device 288 in the expanded configuration, the heat from heated liquid 289 may cause ablation of the walls of the anatomic passageway 102, including the target tissue 284, between the first location 282 and the second location 292. Thus, goblet cells, cilia, and other tissue along the length of the passageway between the first location 282 and the second location 292 may be ablated as the expandable device 288 is moved. In some embodiments, movement of the treatment device from the first location to the second location may be performed manually. In some embodiments, the treatment device may be coupled to a manipulator of a robot-assisted medical system (e.g., system 500) and movement of the treatment device from the first location to the second location may be performed by actuation of a manipulator.

In some embodiments and for some treatment plans, the expandable device may be applied at a plurality of discrete locations along the anatomic passageway rather than being drawn along the passageway in an expanded configuration. At an optional process 260, the treatment device may be collapsed at the first location after heat is applied to the wall passageway. For example, the expandable device 288 may be collapsed from the expanded configuration to an unexpanded configuration in which the expandable device generally does not radially contact the anatomic passageway 102. When the expandable device 288 is collapsed, some or all of the liquid 289 may be evacuated from the expandable device.

At an optional process 262, the collapsed treatment device may be moved from the first location to a second sequential location. For example, the expandable device 288 in the collapsed configuration may be moved from the first location 282 to the second location 292. While the expandable device 288 is moving, the expandable device may be insufficiently heated to ablate tissue and may have little or no contact with the walls of airway 102, thus the length of the airway between the first and second locations may not be ablated.

At an optional process 264, the treatment device is expanded at the second location. For example, the medical instrument 280 may be re-expanded to an expanded configuration and may be refilled with the liquid 289. The liquid may be heated to cause ablation of the airway 102 at the second location 292. In some embodiments, the expandable device 288 may be placed in a plurality of airway regions, such as in fourth to fifteenth generation airways, to create sequential heat delivery to various locations of the targeted anatomy.

The method 200, described above, may use the heated treatment device to compress adjacent vasculature to restrict heat dissipating blood flow while the heat is applied to occlude the vasculature. The method 250, described above, may use the heated treatment device to ablate the bronchial passageway while avoiding ablation of the adjacent vasculature or providing only minor ablation to the adjacent vasculature. Any of these methods, either alone or in combination, may be used to effectively treat a variety of disease states. The depth of ablation and therefore the anatomical structures (e.g., bronchial passageway, bronchial artery, pulmonary artery, etc.) occluded by the ablation may be controlled based on the duration of application, the temperature of the expanded and heated device, the extent of expansion of the expandable device, and the determined distance between the heated, expandable device and the anatomical structure.

FIG. 7A-1 illustrates the disease state of chronic bronchitis in the bronchus 115. The bronchus 115 is surrounded by the vasculature 119, including pulmonary artery 138 and bronchial arteries 140. Chronic bronchitis may cause a proliferation of hypersecreting goblet cells and damaged cilia that may lead to excess mucus. The excess mucus may lead to increased respiratory infections, reduced lung function, and reduced quality of life. Goblet cells may reside at a depth of approximately 0.05 mm to 0.10 mm depth. A heated balloon catheter system (e.g., medical instrument 100) may ablate cilia and hypersecreting goblet cells while preserving the cellular matrix and allowing the regrowth of healthier goblet cells and cilia. The healthy regrowth may reduce mucus production, improve lung function, and improve quality of life. Chronic bronchitis airways may form pleats 700. As shown in FIG. 7A-2, an expandable device 702 (e.g. expandable device 132, 288) expanded in lumen 122 of the bronchus 115 may dilate and splay open the pleats 700, exposing the valleys of the pleats and therefore more hypersecreting goblet cells to the heat from the expandable device 702 and thereby increasing the effectiveness of the treatment compared to treatments that do not dilate the airway.

The use of these systems and methods for treatment of chronic bronchitis may be effective in, for example, the third to fifth generation bronchi and may be used in any or all of the lobes of the lung. To treat chronic bronchitis, the heated balloon catheter may be deployed multiple times during a treatment procedure. For example, ablation may be performed between approximately 9 and 54 times during a treatment procedure and at different locations. For treatment of chronic bronchitis, a temperature of the heated balloon may be approximately 60 to 70 degrees Celsius during treatment. The duration of each deployment of the balloon may be, for example, approximately 5 to 15 seconds and may cause ablation to a depth of approximately 0.05 mm to 0.10 mm. For treatment of chronic bronchitis, the bronchial dilation may be approximately 20% or within a range between approximately 10% to 30% (versus the un-dilated passageway) for suitable pleat exposure. The expanded balloon may temporarily compress the bronchial arteries, however by controlling airway ablation depth, heat does not reach the bronchial or pulmonary arteries and therefore no thermal damage or subsequent permanent occlusion may occur to the arteries. Additionally, by limiting the ablation depth to the cilia and goblet cells, ablation of the airway intima is avoided which can result in unwanted occlusive neo-intima hyperplasia and reduction of airway diameter from collagen shrinkage.

FIG. 7B-1 illustrates the disease state of bronchiectasis in the bronchus 115. Bronchiectasis may result in enlarged bronchial pockets and damaged cilia. This may cause excess mucus which may lead to increased respiratory infections, reduced lung function, and reduced quality of life. A heated balloon catheter system (e.g., medical instrument 100) may ablate cilia and the bronchial wall, resulting in a regrowth of healthier cilia and a reduction in bronchial pocket size due to collagen shrinkage and neo-intima hyperplasia. Mucus loads in the bronchial passageway may be reduced and mucus clearance may increase, resulting in a decrease in respiratory infections, improved lung function, and improved quality of life. When treating bronchiectasis, ablation of the collagen in the airways may induce a tightening of the airway walls to reduce over-dilation associated with the disease. Further, ablation of the biofilm, in bronchial pockets that create a nidus for infections, may reduce exacerbations. As shown in FIG. 7B-2, an expandable device 704 (e.g. expandable device 132, 288) may be expanded in lumen 122 of the bronchus 115 and may dilate the lumen 122.

The use of these systems and methods for treatment of bronchiectasis may be effective in, for example, the third to fifth generation bronchi and may be used in any of three lobes of the lung during a procedure. To treat bronchiectasis, the heated balloon catheter may be deployed multiple times during a treatment procedure. For example, ablation may be performed between approximately 3 and 21 times during a treatment procedure and at different locations. For treatment of bronchiectasis, a temperature of the heated balloon may be approximately 70 to 90 degrees Celsius during treatment. The duration of each deployment of the balloon may be, for example, approximately 5 to 20 seconds and may cause ablation to a depth of approximately 0.30 mm to 1.0 mm. For treatment of bronchiectasis, the heated balloon may be expanded in the bronchial passageway but may provide little (e.g. approximately 5% to 20% dilation) or no dilation of the passageway and little or no compression of adjacent arteries. The duration, temperature, and depth of ablation may be selected based on bronchial wall thickness. The bronchial wall depth may vary in thickness from approximately 0.3 mm to 1.0 mm and may be determined from imaging data, such as intra-operative or pre-operative CT data. Once the wall thickness is determined, the time to ablate through the bronchial wall but not through the bronchial arteries may be determined.

Ablation of a radial segment of a bronchial passageway may also be used to treat hemoptysis which may result from various disease states, including bronchiectasis. Ablation of a hemorrhaging bronchial artery, often caused by bronchiectasis, may be life-saving. A heated dilated balloon (e.g. expandable device 132, 288, 704) may include insulated and non-insulated (e.g., conductive) portions or radial rings that restrict ablation to radial segments of a bronchial passageway. Once the hemorrhage is located, the heated dilated balloon may be applied to the bronchial passageway where the ruptured bronchial artery resides. Dilation may stop blood flow by compressing ruptured artery, and the application of heat may shrink and occlude the artery via thrombosis, with subsequent permanent occlusion via neointima hyperplasia of artery. By ablating only a radial segment of the air passageway, complete occlusion of the passageway may be averted and the airway remains patent. This may allow the segment of lung distal to the ablation to remain functional.

FIG. 7C-1 illustrates the disease state of emphysema. Emphysema may result in heterogeneously distributed hyperinflation of the alveoli 116. These hyperinflated segments of the lung may have poor gas exchange and may encroach upon healthier regions of alveoli. Reducing these poorly functioning hyperinflated segments of lung may improve lung function and quality of life. A heated balloon catheter system (e.g., medical instrument 100) may ablate the bronchial wall 124 resulting in collagen shrinkage and neo-intima hyperplasia. This may lead to occlusion of the passageway 122 and subsequent lung volume reduction of that hyperinflated segment. This may lead to improved lung function and quality of life. As shown in FIG. 7C-1, an expandable device 706 (e.g. expandable device 132, 288) may be expanded in lumen 122 of the bronchus 115.

The use of these systems and methods for treatment of emphysema may be effective in, for example, the third to fifth generation bronchi and may be used in approximately 3 to 6 segments during a procedure. To treat emphysema, the heated balloon catheter may be deployed multiple times during a treatment procedure. For example, ablation may be performed between approximately 3 and 21 times during a treatment procedure and at different locations. For treatment of emphysema, a temperature of the heated balloon may be approximately 70 to 90 degrees Celsius during treatment. The duration of each deployment of the balloon may be, for example, approximately 5 to 20 seconds and may cause ablation to a depth of approximately 0.30 mm to 1.0 mm. For treatment of emphysema, the heated balloon may be expanded in the bronchial passageway but may provide little (e.g. approximately 5% to 20% dilation) or no dilation of the passageway and little or no compression of adjacent arteries. Although the bronchial arteries 140 may be compressed by dilation of the bronchial wall, they may not be ablated because the depth of ablation may extend only through to the adventitia. Therefore, in some embodiments, no thermal damage or subsequent permanent occlusion may occur to the arteries. The duration, temperature, and depth of ablation of the passageway may be selected based on bronchial wall thickness. The bronchial wall depth may vary in thickness from approximately 0.3 mm to 1.0 mm and may be determined from imaging data, such as intra-operative or pre-operative CT data. Once the wall thickness is determined, the time to ablate through the bronchial wall but not through the bronchial arteries may be determined. As shown in FIG. 7C-2, the bronchus 115 may become occluded after the ablation. This may result in reduced hyperinflation of lung segments, reduced lung volume of, for example 300 to 1000 cc, and reduced encroachment on adjacent alveoli.

As shown in FIG. 7D-1, emphysema may also or alternatively be treated using the expandable device 706 to both ablate the passageway 122 of the bronchus 115 and ablate the adjacent bronchial arteries 140. In some embodiments, the pulmonary artery 138 may not be ablated by the heat from the expandable device 706. The ablation of the bronchial arteries may lead to a partial infarction of the hyperinflated segment, resulting in additional volume reduction through alveoli infarction. In this embodiment, ablation may be performed between approximately one and six times during a treatment procedure. For treatment of emphysema that includes ablation of the bronchial arteries, a temperature of the heated balloon may be approximately 85 to 95 degrees Celsius during treatment. The duration of each deployment of the balloon may be, for example, approximately 20 to 120 seconds and may cause ablation to a depth of approximately 1.0 mm to 3.0 mm. For treatment of emphysema in this embodiment, the heated balloon may be expanded in the bronchial passageway to dilate the passageway between approximately 10% and 50% to compress the bronchial arteries. The compression of the bronchial arteries may eliminate the heat dissipation from blood flow in the bronchial arteries. The extent of the passageway dilation may be insufficient to compress the pulmonary artery. Because blood may continue to flow through the uncompressed pulmonary artery, heat from the balloon may be dissipated by the blood, preventing ablation of the pulmonary artery. As shown in FIG. 7D-2, the bronchus 115 may become occluded after the ablation. This may result in reduced hyperinflation of lung segments, partial infarction of the parenchyma, reduced lung volume of, for example 300 to 1000 cc, and reduced encroachment on adjacent alveoli.

FIG. 7E-1 illustrates the disease state of lung tumor. As described above, full ablation of the bronchus (e.g., the airway), bronchial arteries, and the pulmonary artery may be used to treat one or more lung tumors. Lung tumors are often irregular in shape and composition making them difficult to completely ablate by the application of direct heat to the tumor. Failure to completely necrose all tumor cells may result in regrowth of the tumor. Creating a complete infarction of the lung segment where the tumor resides may cause complete necrosis of the tumor and may result in the complete elimination of the tumor via the tissue replacement process. As shown in FIG. 7E-1, an expandable device 708 (e.g. expandable device 132, 288) may be expanded in lumen 122 of the bronchus 115. The expandable device 708 may ablate the bronchial wall 124, bronchial arteries 140 and pulmonary artery 138, resulting in complete infarction of the segment where the tumor 127 resides. This method of tumor death may include destruction of the tumor margin, which is has been associated with low recurrence rates for surgical resection. Ablation of the bronchial wall 124 may result in collagen shrinkage and neo-intima hyperplasia. This may lead to airway occlusion which blocks a first source of oxygen for the lung segment. Ablation of the bronchial arteries 140 may occlude the bronchial arteries by neointimal hyperplasia and collagen shrinkage, thereby blocking a second source of oxygen (oxygenated blood) to the lung segment. Ablation of the pulmonary artery 138 may occlude the arteries by neointimal hyperplasia and collagen shrinkage, thereby blocking a third source of oxygen (oxygenated blood) to the lung segment. This technique for non-direct thermal ablation necrosis of the tumor may maximize the abscopal effect by not altering the DNA of the cancer cells.

The use of these systems and methods for treatment of a lung tumor may be effective in, for example, the fourth to sixth generation bronchi. To treat a tumor, the heated balloon catheter may be deployed multiple times during a treatment procedure. For example, ablation may be performed between approximately one and three times during a treatment procedure and at different locations. For treatment of a lung tumor, a temperature of the heated balloon may be approximately 90 to 95 degrees Celsius during treatment. The duration of each deployment of the balloon may be, for example, approximately 120 to 300 seconds and may cause ablation to a depth of approximately 3.0 mm to 5.0 mm. For treatment of a tumor, the bronchial passage may be dilated by 50 to 100% to compress the bronchial arteries 140 and pulmonary artery 138. Compression of the arteries may eliminate the heat dissipation from arterial blood flow. The duration, temperature, and depth of ablation of the passageway may be selected based on bronchial wall thickness. The bronchial wall depth and pulmonary artery radial distance may vary in thickness from approximately 0.3 mm to 1.0 mm) and may be determined from imaging data, such as intra-operative or pre-operative CT data. Once the wall thickness is determined, the time to ablate through the bronchial wall but not through the bronchial arteries may be determined. As shown in FIG. 7E-2, the bronchus 115, the bronchial arteries 140, and the pulmonary artery 138 may become occluded after the ablation. This may result in blockage of oxygen to the alveoli in the segment where the tumor 127 is located which may cause a complete infarction of the disease segment, tumor necrosis, and biological removal of the necrotic tissue from the body.

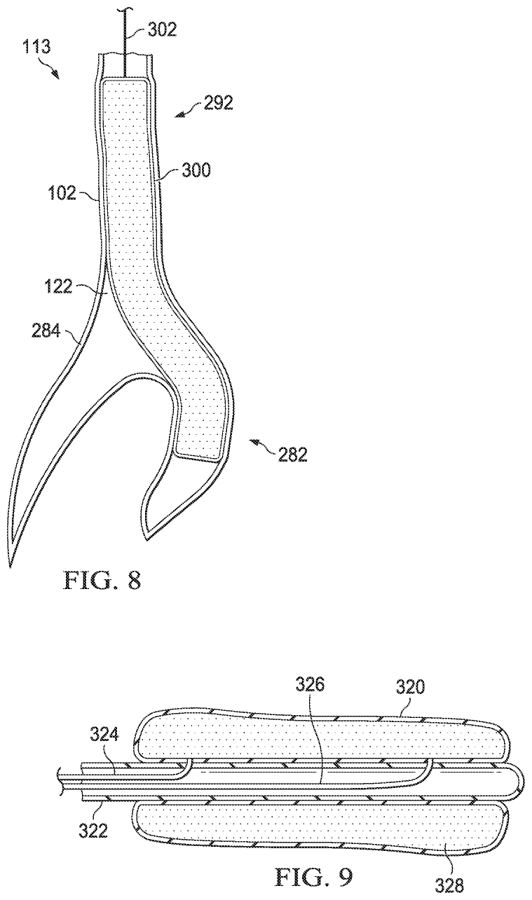

FIGS. 8-14 illustrate various embodiments of expansion or expandable devices that may be used to perform any of the methods described above. These expandable devices may be incorporated into the medical instrument system 100 which may be referred to as a heated balloon catheter system.

FIG. 8 illustrates an expandable device 300 (e.g., expandable device 132, 288) coupled to a distal end of a flexible catheter 302. In this embodiment, the expandable device 300 is an elongated balloon that may extend from the first location 282 to the second location 292, ablating the surface (and optionally subsurface) along the wall of airway 102 from the first location 282 to the second location 292. In some embodiments, the expandable device 300 may have a length between approximately 5 and 60 mm and a diameter between approximately 0.5 and 5 mm. In some embodiments, the expandable device may span a plurality of airway generations. In some embodiments, the temperature of the fluid in the expandable device 300 may be controlled at a temperature between approximately 38 and 99 degrees Celsius.

FIG. 9 illustrates an expandable device 320 (e.g., expandable device 132, 288, 300) coupled to a distal end of a flexible catheter 322. In this example, fluid 328 may be heated proximally of the expandable device 320 and may be circulated into the expandable device through a fluid inlet 324 and out of the expandable device through a fluid outlet 326. In some embodiments, the fluid may be a non-compressible liquid or gel.

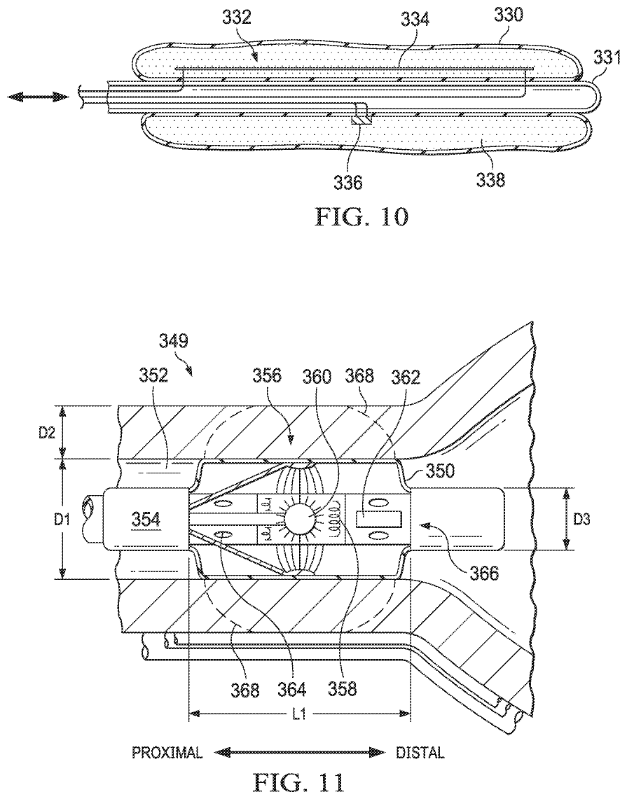

FIG. 10 illustrates an expandable device 330 (e.g., expandable device 132, 288, 300) coupled to a distal end of a flexible catheter 331. A heating system 332 (e.g. heating system 134, 290) is located in the expandable device 330 or along a distal portion of the catheter 331 and comprises a heating element 334 and a sensor 336. The heating element 334 may include, for example, a radiofrequency heating element, a resistive heating element, a microwave heating element, an ultrasonic heating element, a magnetic heating element, or a light/laser-based heating element. The heating element 334 may heat a fluid 338 that flows within the expandable device 330. The sensor 336 may be, for example, a thermocouple and may sense the temperature of the fluid 338 and provide feedback for controlling power to the heating element 334. Based on the sensed temperature, power to the heating element 334 may cycled to control the temperature of the expandable device 330.

FIG. 11 illustrates a medical instrument system 349 including an expandable device 350 (e.g., expandable device 132, 288) within an anatomic passage 352 (e.g., passageway 102). The expandable device 350, such as a balloon, may have a generally cylindrical shape and may be coupled to a distal end of a flexible catheter 354. The system 349 also includes a heating system 356 that includes bipolar radio-frequency electrodes with an electrode 358 positioned within the expandable device 350, for example on a distal portion of the catheter 354 and with an electrode 360 positioned on a surface of the expandable device. In some embodiments, a plurality of electrodes may be placed within the expandable device and a plurality of electrodes may be placed on the surface of the expandable device. The heating system 356 may also include a temperature sensor 362 that may be substantially similar to sensor 336. Fluid may flow into the expandable device 350 through fluid inlet 364 and may flow out of the expandable device through fluid outlet 366. In some embodiments, the expandable device 350 may have a length L1 of between approximately 5 and 60 mm. In some embodiments, the temperature of the fluid in the expandable device 350 may be controlled at a temperature between approximately 60 and 99 degrees Celsius. In some embodiments, a diameter D1 of anatomic passageway 352 and the expandable device 350 may be between approximately 2 and 10 mm and in some embodiments may be approximately 4 mm. In some embodiments, an ablation zone 368 may have a depth D2 of between approximately 1 and 5 mm. In some embodiments, the catheter 354 may have a diameter D3 that may be approximately 1.8 mm. In some embodiments, the system 349 may be suited for treatment of emphysema and may induce airway occlusion and an inflammatory response that induces lung volume distal of the ablation zone 368.

When one electrode is placed on the surface of the expandable device and one is placed on the catheter, as shown in the embodiment of FIG. 11, the liquid (e.g. a saline solution) may become the conductor of the RF energy, and the current running through the liquid will heat the liquid. In alternative embodiments, electrodes may be omitted from the surface of the expandable device and instead at least two electrodes may be spaced apart along the catheter. In this embodiment, the current may be conducted through the liquid to heat the liquid or the current may run from one electrode to the other along the catheter shaft to heat the shaft and thus the surrounding liquid. Whether the current conducts through the catheter or through the liquid depends on which has the least resistive path. In any of these embodiments, the heated liquid may conduct the thermal energy to the tissue with the electrodes heating the liquid but not directly heating the adjacent tissue. In alternative embodiments, the RF energy may be applied so that the current travels through the tissue to directly heat the tissue.

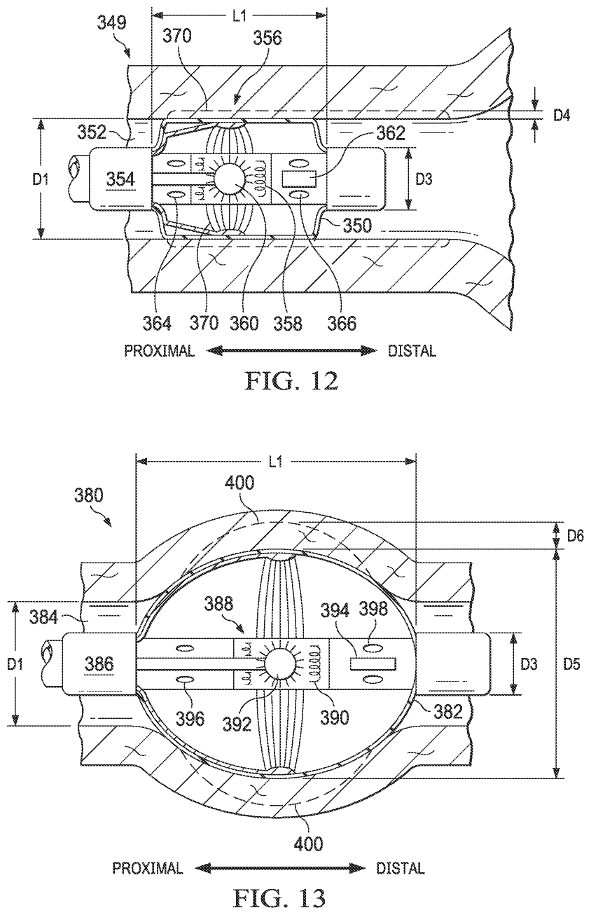

FIG. 12 illustrates the system 349 for use in generating an ablation zone 370 that may be shallower than the ablation zone 368 due to fluid temperature, duration of application, and/or pressure. In this embodiment, the ablation zone may have a depth D4 between approximately 0.1 and 1.5 mm and may be suitable for treatment of chronic bronchitis, particularly the ablation of goblet cells.

FIG. 13 illustrates a system 380 including an expandable device 382 (e.g., expandable device 132, 288) within an anatomic passage 384 (e.g., passageway 102). The expandable device 382, such as a balloon, has a generally oval shape and is coupled to a distal end of a flexible catheter 386. The system 380 also includes a heating system 388 that includes bipolar radiofrequency electrodes with an electrode 390 positioned within the expandable device 382, for example on a distal portion of the catheter 386 and with an electrode 392 positioned on a surface of the expandable device. In some embodiments, a plurality of electrodes may be placed within the expandable device and a plurality of electrodes may be placed on the surface of the expandable device. The heating system 388 may also include a temperature sensor 394 that may be substantially similar to sensor 336. Fluid may flow into the expandable device 382 through fluid inlet 396 and may flow out of the expandable device through fluid outlet 398. In some embodiments, the expandable device 382 may have a length L1 of between approximately 5 and 60 mm. In some embodiments, the temperature of the fluid in the expandable device 382 may be controlled at a temperature between approximately 60 and 99 degrees Celsius. In some embodiments, a diameter D5 of a bronchial pocket and the expandable device 382 may be between approximately 2 and 10 mm. In some embodiments, an ablation zone 400 may have a depth D6 of between approximately 1 and 6 mm. In some embodiments, the catheter 386 may have a diameter D3 that may be approximately 1.8 mm. In some embodiments, the system 380 may be suited for treatment of bronchiectasis by ablating collagen in the passageway 384 and destroying biofilm within the bronchial pocket that may create a nidus for infection.

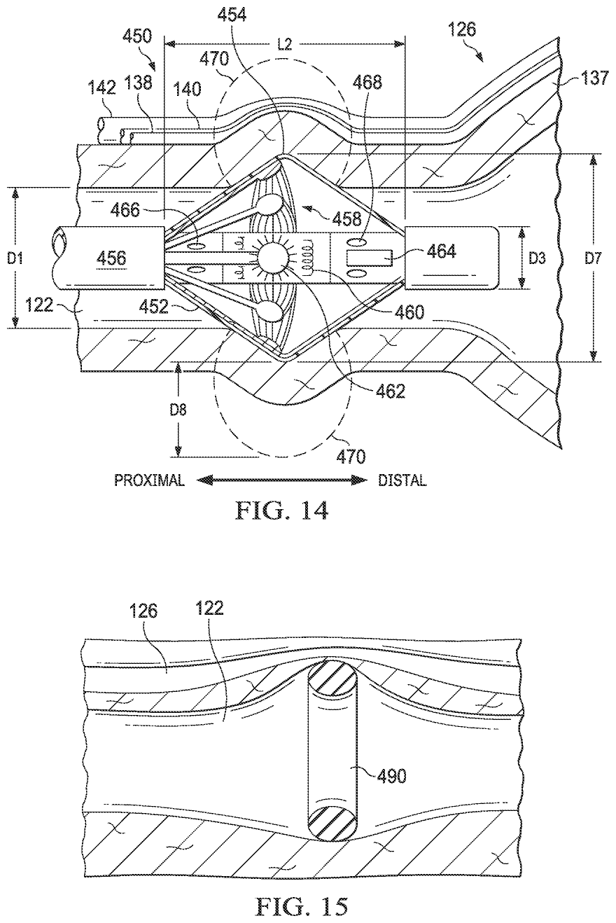

FIG. 14 illustrates a system 450 that may be similar to medical instrument system 100 with details or differences as identified and may be used to perform the method 200. The system 450 may include an expandable device 452, such as a balloon, that has a generally diamond-shaped cross-section including a ridge 454 when expanded to an expanded configuration. The expandable device 452 may be coupled to a distal end of a flexible catheter 456. The system 450 also includes a heating system 458 that includes bipolar radiofrequency electrodes with an electrode 460 positioned within the expandable device 452, for example on a distal portion of the catheter 456 and with an electrode 462 positioned on a surface of the expandable device. In some embodiments, a plurality of electrodes may be placed within the expandable device and a plurality of electrodes may be placed on the surface of the expandable device. The heating system 458 may also include a temperature sensor 464 that may be substantially similar to sensor 336. Fluid may flow into the expandable device 452 through fluid inlet 466 and may flow out of the expandable device through fluid outlet 468. In some embodiments, the expandable device 452 may have a length L2 of approximately 7 mm and a diameter D7 at the ridge 454 of approximately 6.5 mm. In alternative embodiments, the expandable device may have other shapes including cone-shaped, double-cone shaped, asymmetrical, or multi-lobed. In some embodiments, an ablation zone 470 may have a depth D8 of between approximately 3 and 5 mm. In some embodiments, an inner diameter D1 of lumen 122 may be approximately 4 mm. In some embodiments, the catheter 456 may have a diameter D3 that may be approximately 1.8 mm.

In some embodiments, the system 450 may be suited for treatment of distal lung tumors or emphysema. For example, the system 450 may reduce blood flow to target tissue at a second, more distal, location as described in method 200. For example, the occlusion of the pulmonary artery 138 may cause apoptosis of distal (e.g., downstream) cells and infarction of the tissue distal of the first location supplied by the artery 138. Over time, coagulative necrosis of the infarcted tissue, including a target tumor, and the surrounding margin will occur, followed by macrophage removal of the necrotic tissue. In some embodiments, the system 450 may be applied at a plurality of locations to infarct a larger downstream region or to target regions that may have collateral blood flow.

FIG. 15 illustrates an expandable device 490 that may be positioned within the lumen 122. The expandable device 490, which may be an expandable ring, may be inserted through the lumen 122 in a compressed configuration and may be expanded to an expanded configuration to compress the vasculature 119 as described in process 204 of method 200. The expandable device 490 may remain in the expanded configuration as heat is applied from within the lumen 122 to occlude the vasculature 119. Heat may be applied using any of a variety of techniques includes a heated expandable device, a flowable heated liquid, an RF or MW ablation probe, or other heat delivery tool. In some embodiments, the ring may have a narrow profile of approximately 1 mm to provide a sharp pressure zone. In alternative embodiments, other mechanical spreader at the tip of a catheter or other type of mechanical stretching tool may be used within the lumen 122 to mechanically occlude the vasculature 119.

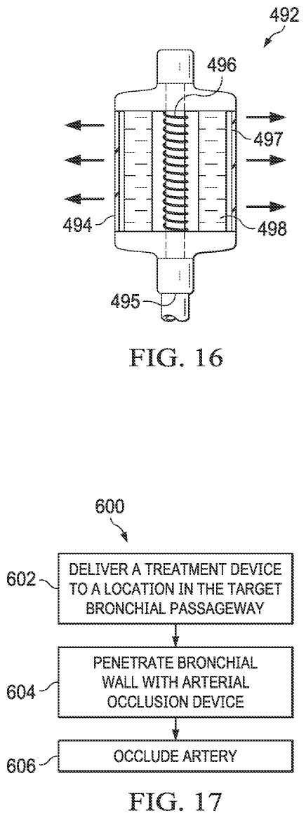

FIG. 16 illustrates a system 492 including an expandable device 494 (e.g., expandable device 132, 288) that may be used within an anatomic passageway (e.g., passageway 102). The expandable device 494, such as a balloon, has a generally cylindrical shape and is coupled to a distal end of a flexible catheter 495. The system 492 also includes a heating system 496 that includes a resistive heating element or bipolar radiofrequency electrodes within the expandable device 494 to heat a fluid 498. An RF shell 497 may be disposed on a surface of the expandable device 494. The RF shell may be, for example, a metallic plated or painted electrode. The RF shell 497 may be monopolar so that heat from the heated fluid 498 is projected radially outward from the expandable device 494. The system 492 may create a larger region of ablation, capable of fully ablating an adjacent artery, as compared to some embodiments of a heated expandable device without an RF shell.

In any of the instrument systems described above, the liquid may be heated by the heating system at the distal end portion of the treatment device before being released from the distal end portion of the treatment device into the patient anatomy.

In any of the instrument systems described above, the liquid may be circulated to improve heat distribution. For example, the liquid may be circulated by an impeller, by a cycle of injection and aspiration, by a vibration of the heating system in the expandable device, by contracting and inflating the expandable device and/or by rotating, oscillating or otherwise moving the heating system or a component within the expandable device. In some embodiments, portions of the heating system may be vibrated to reduce bubble formation on the heating system.

In any of the instrument systems described above, the expandable device may be balloon reinforced with, for example, a braiding or a metallic coating to sustain the balloon through temperatures which would otherwise degrade or change the mechanical properties of the balloon.

In some embodiments, reliable lung tumor removal may be achieved by occlusion of the bronchial lumen, the pulmonary artery and the bronchial artery. In some embodiments, a heated balloon or diffuse heated liquid treatment may occlude the bronchial lumen, the pulmonary artery, and the bronchial artery. Sometimes, however, these treatments alone may not be sufficient for full occlusion of all structures. For example, the elastic limit of the bronchial wall may be reached before a heated dilated balloon may cause collapse of the pulmonary artery. An incomplete pulmonary artery collapse may permit continued blood flow and heat dissipation associated with the blood flow. This continued blood flow and heat dissipation may limit the extent of arterial ablation and the subsequent neointimal hyperplasia and may result in a partial infarction or no infarction at all.

In some embodiments, a total acute collapse of the pulmonary artery, via the heated dilated balloon, may not be required to achieve total permanent occlusion of the pulmonary artery. This is because heat to the inner diameter of the artery closest to the heated dilated balloon will cause platelet activation and aggregation which may be sufficient to create acute occlusion. Acute occlusion may create hemostasis which eliminates heat dissipating blood flow through the pulmonary artery, allowing for heat conduction through the static blood and resulting in ablation of the entire circumference of the pulmonary artery. A complete circumferential ablation of the pulmonary artery may maximize the potential for complete permanent neo-intimal hyperplasia occlusion, resulting in a maximum propensity for parenchymal infarction. If, however, platelet activation and aggregation of a partially occluded pulmonary artery from the heated balloon device does not create acute hemostasis, there may be blood flow in the pulmonary artery which could act as a heat dissipater potentially prohibiting ablation of the entire circumference of the pulmonary artery. Failure to ablate the entire circumference of the pulmonary artery may reduce the possibility of permanent pulmonary occlusion via neo-intimal hyperplasia.

Various systems and methods are described below for using the bronchial passageway as a launch point from which a tool may be inserted into an adjacent artery to directly occlude the artery. Such systems and techniques may also be used to arrest bleeding of a ruptured artery. FIG. 17 illustrates a method 600 that may be used to occlude vasculature, including the pulmonary artery that extends along a bronchial passageway to aid in the infarction, necrosis, and subsequent lung tumor removal. At a process 602, a medical instrument may be delivered to a location in a target bronchial passageway. As previously described, a medical instrument may be coupled to a robot-assisted manipulator and navigated to the target location or may be manually positioned at the target location. At a process 604, the medical instrument may penetrate the bronchial wall with an arterial occlusion device. The arterial occlusion device may be moved from the bronchial passageway, through the bronchial wall, and into the artery (e.g., the pulmonary artery) adjacent the bronchial passageway. At a process 606, the arterial occlusion device may occlude the artery by creating an acute clot that prevents blood flow. The stasis of blood may prevent bleeding from the artery into airway and may allow for conduction of heat through the clot providing for complete circumferential ablation of the artery. This may maximize the potential for complete permanent neointimal hyperplasia occlusion. Optionally, the risk of bleeding between puncture site of the airway and the pulmonary artery may be reduced by RF cauterization, an application of fibrin glue, the application of a heated dilated balloon tamponade, the application of an unheated dilated balloon tamponade. Optionally, the other methods such as the localized heated dilation balloon ablation method 200 or the diffuse heated liquid ablation method of incorporated by reference PCT Application (Docket No. P02303-WO) filed Jul. 7, 2020, titled “Systems and Method for Diffuse Endoluminal Thermal Liquid Treatment.” may be further used to perform ablation of the pulmonary artery, the bronchial artery, and/or the bronchial passageway.

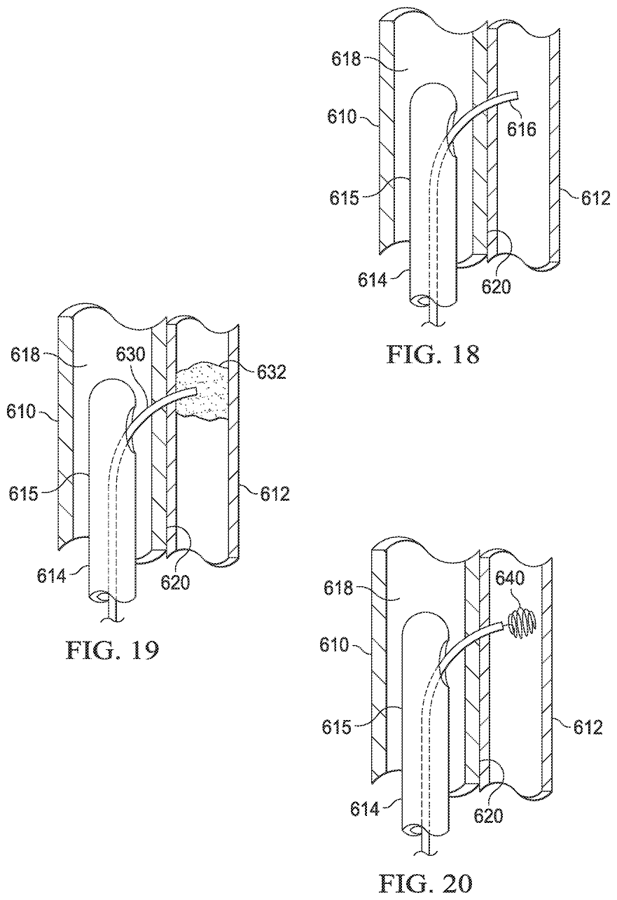

FIGS. 18, 19, 20, 21, and 22A-B illustrate examples of arterial occlusion devices that may be used to perform the method 600. FIG. 18 illustrates a bronchus 610 having an adjacent pulmonary artery 612. A catheter 614 may deliver an arterial occlusion device 616 into a lumen 618 of the bronchus 610. The location of the pulmonary artery 612 relative to the bronchus 610 may be determined using, for example, intra-operative imaging. The catheter 614 may be rotated to the direction of the pulmonary artery 612 and the arterial occlusion device 616 may be advanced through a distal opening 615 in a side of the catheter, though a bronchial wall 620, and into the pulmonary artery 612. In this embodiment, the arterial occlusion device may be an RF wire. After placement in the pulmonary artery 612, the RF wire may be activated to cause platelet aggregation and the formation of an occlusive clot. With blood flow in the pulmonary artery 612 blocked by the blood clot, the RF wire may be removed from the artery and the catheter 614. After the blood clot is formed and the heat dissipation effect of the blood flow is terminated by the clot, a heated dilation balloon ablation method may be performed to locally heat and ablate the pulmonary artery, without fully collapsing the artery.

FIG. 19 illustrates the bronchus 610 having the adjacent pulmonary artery 612. A catheter 614 may deliver an arterial occlusion device 630 into a lumen 618 of the bronchus 610. The location of the pulmonary artery 612 relative to the bronchus 610 may be determined using, for example, intra-operative imaging. The catheter 614 may be rotated to the direction of the pulmonary artery 612 and the arterial occlusion device 630 may be advanced though the bronchial wall 620 into the pulmonary artery 612. In this embodiment, the arterial occlusion device may be a hollow needle catheter for delivery of an occlusive material 632. After placement in the pulmonary artery 612, the needle catheter may deliver the occlusive material into the artery 612 to create a clot. The occlusive material may be injected and may include a fibrin glue, cyanoacrylate, liquid comprising microspheres, alcohol, heated liquid such as water, collagen, tranexamic acid, thromboxane, adenosine diphosphate, and/or a gelatin sponge. The occlusive material may cause platelet aggregation resulting in a blood clot or the material itself may cause an occlusive clot. After the blood clot is formed and the heat dissipation effect of the blood flow is terminated by the clot, a heated dilation balloon ablation method may be performed to locally heat and ablate the pulmonary artery, without fully collapsing the artery.

FIG. 20 illustrates the catheter 614 for delivery of an arterial occlusion device 640 into the lumen 618 of the bronchus 610. The location of the pulmonary artery 612 relative to the bronchus 610 may be determined using, for example, intra-operative imaging. The catheter 614 may be rotated to the direction of the pulmonary artery 612 and the arterial occlusion device 640 may be advanced though the bronchial wall 620 into the pulmonary artery 612. In this embodiment, the arterial occlusion device may include a delivery catheter for delivery of an occlusive coil. The coil may be formed of an elastic material, such as nitinol or an elastomer, that may be straightened for deployment through the delivery catheter and may return to a coiled shape after emerging from the delivery catheter and being inserted into the artery 612. The coil may cause platelet aggregation resulting in a blood clot. After the blood clot is formed and the heat dissipation effect of the blood flow is terminated by the clot, a heated dilation balloon ablation method may be performed to locally heat and ablate the pulmonary artery, without fully collapsing the artery.

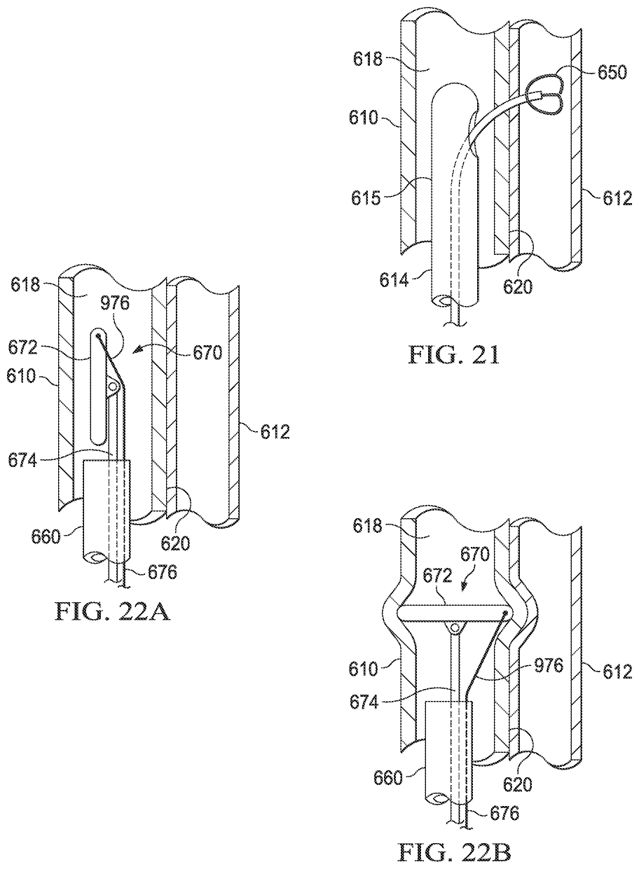

FIG. 21 illustrates the catheter 914 for delivery of an arterial occlusion device 650 into the lumen 618 of the bronchus 610. The location of the pulmonary artery 612 relative to the bronchus 610 may be determined using, for example, intra-operative imaging. The catheter 614 may be rotated to the direction of the pulmonary artery 612 and the arterial occlusion device 650 may be advanced though the bronchial wall 620 into the pulmonary artery 612. In this embodiment, the arterial occlusion device may include a delivery catheter for delivery of a balloon. The balloon may be a silicone balloon extended over an opening in the delivery catheter. The balloon may be inflated with a liquid that has a high viscosity, such as a fibrin glue. Once inflated, the delivery catheter may be withdrawn from the balloon, leaving the balloon behind to occlude the artery. After the balloon is inflated and the heat dissipation effect of the blood flow is terminated by the balloon, a heated dilation balloon ablation method may be performed to locally heat and ablate the pulmonary artery, without fully collapsing the artery.

FIGS. 22A and 22B illustrate a catheter 660 for delivery of an arterial occlusion device 670 into the lumen 618 of the bronchus 610. In this embodiment, the arterial occlusion device 670 may include an occlusion bar 672 pivotally coupled to an RF rod 674. A pullwire 676 coupled to the occlusion bar 672 may be activated by a pull wire 976 to pivot the occlusion bar from an insertion configuration parallel to the catheter 660 (as in FIG. 22A) to an occlusion configuration (as in FIG. 22B) in which the occlusion bar 672 is approximately transverse to the catheter 660. The occlusion bar 672 in the occlusion configuration may compress the artery 612 to fully or partially occlude blood flow through the artery. RF energy from the RF rod 674 may heat the occlusion bar 672 to cause platelet aggregation resulting in a blood clot in the compressed artery 612. After the blood clot is formed and the heat dissipation effect of the blood flow is terminated by the clot, a heated dilation balloon ablation method may be performed to further locally heat and ablate the pulmonary artery.

In some embodiments, the flow of air through a bronchial passageway may be blocked in addition to or as an alternative to blocking blood flow through the pulmonary artery. Ablation of the airway may induce neointimal hyperplasia and occlusion during the healing process. However, this process may take several days and may be too slow. In some embodiments, a physical airway occlusion device may be inserted after an airway heating procedure. The airway occlusion device may include a plug (e.g. a silicone or plastic plug), a one-way valve device, a glue and/or a foam. The plug may be temporarily placed and may be removed after intended necrosis is complete. Alternatively, it may be absorbed by the body or may be left permanently.

Any of the methods, techniques, or systems described in this disclosure may be used in combination or series with each other or with the methods, techniques, or systems described in the incorporated by reference PCT Application (P02303-WO) filed Jul. 7, 2020, titled “Systems and Method for Diffuse Endoluminal Thermal Liquid Treatment.”

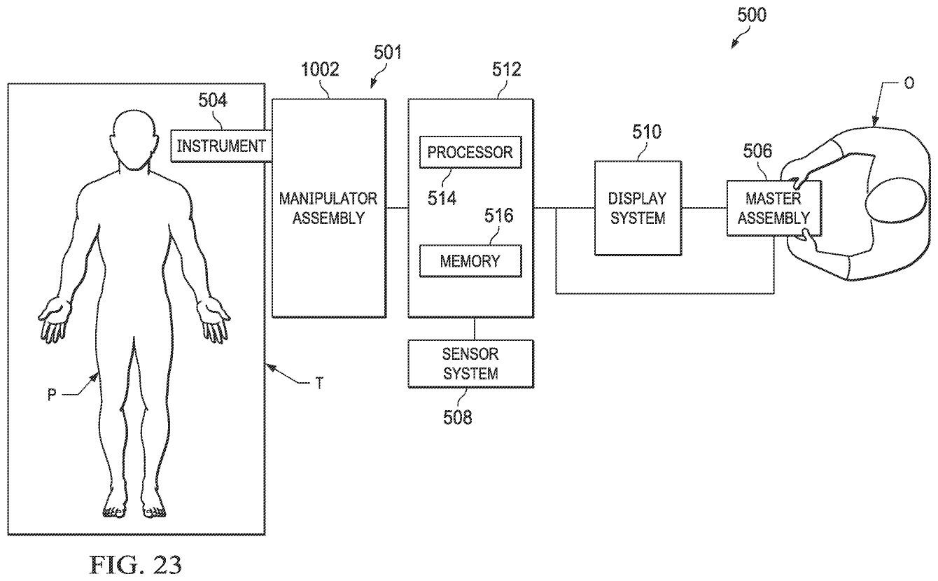

In some embodiments, the systems and methods disclosed herein may be used in a medical procedure performed with a robot-assisted medical system as described in further detail below. As shown in FIG. 23, a robot-assisted medical system 500 may include a manipulator assembly 502 for operating a medical instrument 504 (e.g., medical instrument system 100 or any of the medical instruments with expandable devices described above) in performing various procedures on a patient P positioned on a table T in a surgical environment 501. The manipulator assembly 502 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. A master assembly 506, which may be inside or outside of the surgical environment 501, generally includes one or more control devices for controlling manipulator assembly 502. Manipulator assembly 502 supports medical instrument 504 and may optionally include a plurality of actuators or motors that drive inputs on medical instrument 504 in response to commands from a control system 512. The actuators may optionally include drive systems that when coupled to medical instrument 504 may advance medical instrument 504 into a naturally or surgically created anatomic orifice. Other drive systems may move the distal end of medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes).

Robot-assisted medical system 1100 also includes a display system 510 for displaying an image or representation of the surgical site and medical instrument 504 generated by a sensor system 508 which may include an endoscopic imaging system. Display system 510 and master assembly 506 may be oriented so an operator O can control medical instrument 504 and master assembly 506 with the perception of telepresence. Any of the previously described graphical user interfaces may be displayable on a display system 510 and/or a display system of an independent planning workstation.

The sensor system 508 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument 504. The sensor system 508 may also include temperature sensors, such as the temperature sensors 336, 362, 394, 464.

Robot-assisted medical system 500 may also include control system 512. Control system 512 includes at least one memory 516 and at least one computer processor 514 for effecting control between medical instrument 504, master assembly 506, sensor system 508, and display system 510. Control system 512 also includes programmed instructions (e.g., a non-transitory machine-readable medium storing the instructions) to implement a plurality of operating modes of the robot-assisted medical system including a navigation planning mode, a navigation mode, and/or a procedure mode. Control system 512 also includes programmed instructions (e.g., a non-transitory machine-readable medium storing the instructions) to implement some or all of the processes described in accordance with aspects disclosed herein, including, for example, expanding the expandable device, regulating the temperature of the heating system, controlling insertion and retraction of the treatment instrument, controlling actuation of a distal end of the treatment instrument, receiving sensor information, selecting a treatment location, and/or determining a size to which the expandable device may be expanded.

Control system 512 may optionally further include a virtual visualization system to provide navigation assistance to operator O when controlling medical instrument 504 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. As provided above in the description of method 200, the control system 512 may use a pre-operative image to locate the target tissue and create a pre-operative plan, including an optimal first location for performing bronchial passageway and vasculature occlusion. The pre-operative plan may include, for example, a planned size to expand the expandable device, a treatment time, and/or multiple deployment locations.

In the description, specific details have been set forth describing some embodiments. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions. Not all the illustrated processes may be performed in all embodiments of the disclosed methods. Additionally, one or more processes that are not expressly illustrated in may be included before, after, in between, or as part of the illustrated processes. In some embodiments, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some embodiments are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in embodiments of this disclosure may be implemented in software to execute on a processor of a computer system such as control processing system. When implemented in software, the elements of the embodiments of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes and displays presented might not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term pose refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

Various aspects of the subject matter described herein are set forth in the following numbered examples.

Example 1: A method for treating diseased target tissue causing a chronic lung condition comprising: expanding a treatment device positioned within a first airway at a first location along a length of the first airway; and applying heat using the treatment device, at a temperature and for a time period, for ablation of at least a bronchial wall of the first airway.

Example 2: The method of Example 1, further comprising determining a distance, wherein the distance is based on a thickness of the bronchial wall at the first location.

Example 3: The method of Example 1, further comprising determining a distance, wherein the distance is between an inner surface of the bronchial wall and a plurality of bronchial arteries adjacent the anatomic lumen.

Example 4: The method of Example 2 or 3, wherein determining the distance includes receiving sensor data.

Example 5: The method of Example 2 or 3, wherein determining the distance includes receiving a pre-determined distance.

Example 6: The method of one of Examples 2-5, further comprising expanding the treatment device to an expanded configuration based on the determined distance.

Example 7: The method of one of Examples 2-6, wherein the determined distance provides for decreasing a diameter of at least one of the plurality of bronchial arteries.

Example 8: The method of one of Examples 2-7, wherein the time period is further based on preventing ablation of a plurality of bronchial arteries.

Example 9: The method of one of Example 2-8, wherein the time period is based at least in part on the distance.

Example 10: The method of one of Examples 1-9, wherein the time period based at least in part on the temperature.

Example 11: The method of one of Examples 1-10, wherein expanding the treatment device includes expanding to a deployment size for dilation of the airway by between 5 and 20 percent.

Example 12: The method of one of Examples 1-11, wherein the time period is between 5 and 20 seconds.

Example 13: The method of one of Examples 1-12, wherein the temperature is between 70 and 90 degrees Celsius.

Example 14: The method of one of Examples 1-13, wherein the chronic lung condition is bronchiectasis.

Example 15: The method of one of Examples 1-14, wherein the chronic lung condition is emphysema.

Example 16: The method one of Examples 2 or 3, wherein the determined distance provides for maintaining a diameter a pulmonary artery adjacent the anatomic lumen.

Example 17: The method of any one of Examples 1-10 or 16, wherein expanding the treatment device includes expanding to a deployment size for dilation of the airway by between 10 and 50 percent.

Example 18: The method of any of Examples 1-10 or 16-17, wherein the time period is between 20 and 120 seconds.

Example 19: The method of any of Examples 1-10 or 16-18, wherein the temperature is between 80 and 95 degrees Celsius.

Example 20: The method of any of Examples 1-10 or 16-19, wherein the chronic lung condition is emphysema.

Example 21: The method of any one of Examples 1-10, wherein expanding the treatment device includes expanding to a deployment size for dilation of the airway by between 50 and 100 percent.

Example 22: The method of any of Examples 1-10 or 21, wherein the time period is between 120 and 300 seconds.

Example 23: The method of any of Examples 1-10 or 21-22, wherein the temperature is between 90 and 95 degrees Celsius.

Example 24: The method of one of Examples 2 or 3, wherein the determined distance provides for decreasing a diameter a pulmonary artery adjacent the first airway, wherein the pulmonary artery provides blood flow to the target tissue.

Example 25: The method of claim 24, wherein applying heat using the treatment device occludes the pulmonary artery to prevent blood flow to the target tissue.

Example 26: The method of Example 24 or 25, wherein the first location is at a location along the length of the first airway proximal to the target tissue.

Example 27: The method of one of Examples 24-26, further comprising determining the first location for applying heat to the first airway.

Example 28: The method one of Examples 25-27, further comprising monitoring fluid flow in the pulmonary artery to determine if the pulmonary artery has been occluded.

Example 29: The method of one of Examples 24-28, further comprising determining a second location along the length of the first airway, wherein the target tissue is at the second location.

Example 30: The method of Example 29, further comprising ablating the target tissue at the second location.

Example 31: The method of Example 30, further comprising collapsing the treatment device; repositioning the treatment device at the second location; expanding the treatment device at the second location; and applying heat to ablate the target tissue.

Example 32: The method of one of Examples 24-31, further comprising identifying a blood vessel providing blood to the target tissue, wherein the blood vessel is adjacent a second airway.

Example 33: The method of Example 32, further comprising applying heat to the blood vessel when the treatment device is positioned within the second airway.

Example 32: The method of any one of Examples 1 or 21-33, wherein the target tissue includes a cancerous tumor.

Example 33: The method of Example 32, wherein the target tissue includes a margin surrounding the cancerous tumor.

Example 34: A method for treating a chronic lung condition comprising: expanding a treatment device within an airway to a deployed configuration, wherein the deployed configuration dilates pleats along a wall of the airway; and applying heat using the treatment device, wherein the heat is applied at a temperature and a time period for ablation of goblet cells and cilia within the wall of the airway without damaging bronchial arteries surrounding the airway.

Example 35: The method of Example 34, further comprising determining a distance, wherein the distance is based on a thickness of a bronchial wall of the airway at the first location.

Example 36: The method of Example 34, further comprising determining a distance, wherein the distance is between an inner surface of a bronchial wall of the airway and a plurality of bronchial arteries adjacent the anatomic lumen.

Example 37: The method of Example 35 or 36, wherein determining the distance includes receiving sensor data.

Example 38: The method of Example 35 or 36, wherein determining the distance includes receiving a pre-determined distance.

Example 39: The method of one of Examples 35-38, further comprising expanding the treatment device to an expanded configuration based on the determined distance.

Example 40: The method of one of Examples 35-39, wherein the determined distance provides for decreasing a diameter of at least one of the plurality of bronchial arteries adjacent the airway.

Example 41: The method of Examples 40, wherein the time period is further based on preventing ablation of a plurality of bronchial arteries.

Example 42: The method of one of Example 35-41, wherein the time period is based at least in part on the distance.

Example 43: The method of one of Examples 35-42, wherein the time period based at least in part on the temperature.

Example 44: The method of one of Examples 35-43, wherein expanding the treatment device includes expanding to a deployment size for dilation of the airway by between 10 and 30 percent.

Example 45: The method of one of Examples 35-44, wherein the time period is between 5 and 15 seconds.

Example 46. The method of one of Examples 35-45, wherein the temperature is between 60 and 70 degrees Celsius.

Example 47. The method of one of Examples 35-46, wherein the chronic lung condition is chronic bronchitis.

Example 48: A method for treating bronchiectasis comprising: identifying a ruptured bronchial artery alongside an airway; expanding a device within the airway to a deployed configuration, wherein the deployed configuration compresses the ruptured bronchial artery; and applying heat using the device to occlude the ruptured bronchial artery, wherein the device includes an insulated portion and a conductive portion and wherein the insulated portion is aligned towards the ruptured bronchial artery.

Example 49: The method of one of Examples 1-48, wherein applying the heat includes operating a heating system to heat a liquid.

Example 50: The method of Example 49, wherein the liquid is heated by the heating system at the distal end portion of the treatment device before being released from the distal end portion of the treatment device.

Example 51: The method of Example 49 or 50, wherein the heating system heats the liquid with resistive energy.

Example 52: The method of Example 49 or 50, wherein the heating system heats the liquid with radio-frequency energy.

Example 53: The method of one of Examples 1-48, wherein ablating the target tissue includes applying heat generated by an RF heat source, a microwave heat source, or a resistive heat source.

Example 54: A system for treating target tissue comprising: a catheter including a distal end portion configured for deployment in an anatomic lumen; an expansion device coupled to the distal end portion of the catheter; a processor; and a memory having computer readable instructions stored thereon, the computer readable instructions, when executed by the processor, cause the system to complete the method of one of Examples 1-53.

1. A system for treating target tissue located at a first location along an anatomic lumen, the system comprising:
a catheter including a distal end portion configured for deployment in the anatomic lumen; and
an expansion device coupled to the distal end portion of the catheter, the expansion device having an expanded size in a deployed configuration,
wherein the expansion device in the deployed configuration occludes the anatomic lumen at a second location, different from the first location, along the anatomic lumen and occludes at least one of a plurality of blood vessels adjacent the anatomic lumen to reduce blood flow to the target tissue at the first location.

2. The system of claim 1, wherein the expansion device in the deployed configuration dilates the anatomic lumen and reduces a distance between a wall of the anatomic lumen and the at least one of the plurality of blood vessels.

3. The system of claim 1, wherein the expansion device in the deployed configuration reduces a diameter of the at least one of the plurality of blood vessels.

4. The system of claim 1, wherein the anatomic lumen is an airway; the target tissue includes parenchymal tissue; and the plurality of blood vessels includes a bronchial artery.

5. The system of claim 4, wherein the expansion device in the deployed configuration dilates the airway by between approximately 10 and 50 percent.

6. The system of claim 4, wherein the plurality of blood vessels further includes a pulmonary artery.

7. The system of claim 6, wherein the expansion device in the deployed configuration results in dilation of the airway by between approximately 50 and 100 percent.

8. The system of one of claim 1, wherein the system further comprises a heating system, the heating system configured to heat the expansion device to further occlude the anatomic lumen and the at least one of the plurality of blood vessels.

9. The system of claim 8, wherein the heating system is at the distal end portion of the catheter and wherein the heating system includes at least one of a resistive heating member, an RF heat source, a microwave heat source, an ultrasound heat source, or a laser heat source.

10. The system of claim 8, wherein the expansion device includes a balloon.

11. The system of claim 10, further comprising:
a liquid source providing liquid to fill the balloon to expand the balloon to the deployed configuration.

12. The system of claim 11, wherein the heating system is further configured to heat the liquid.

13. The system of claim 12, wherein the liquid is heated by the heating system at a distal end portion of the catheter after being released into the balloon.

14. (canceled)

15. The system of claim 11, wherein the heating system includes two radio-frequency electrodes and wherein at least one of the radio-frequency electrodes is coupled to a balloon.

16. The system of claim 15, wherein the heating system includes the two radio-frequency electrodes coupled to the catheter.

17. The system of claim 10, wherein the balloon includes a reinforcement member and wherein the reinforcement member is configured to support an inflation of the balloon.

18. The system of claim 17, wherein the reinforcement member includes a braid or a metallic coating.

19. The system of claim 8, wherein the expansion device includes an expandable ring.

20. The system of claim 8, further comprising a processor configured to:
expand the expansion device and regulate the heating system.

21-45. (canceled)

46. The system of claim 20, wherein the expanding of the expansion device is based on a distance between a wall of the anatomic lumen and the at least one of the plurality of blood vessels, an amount of compression of the at least one of the plurality of blood vessels, a diameter of the anatomic lumen, or an amount of blood flow.
